(19) United States
(12) Patent Application Publication
(10) Pub. No.: US 2011/0184248 A1
(43) Pub. Date: Jul. 28, 2011

(54) STORAGE MEDIUM HAVING STORED THEREIN INFORMATION PROCESSING PROGRAM, INFORMATION PROCESSING DEVICE, INFORMATION PROCESSING METHOD, AND INFORMATION PROCESSING SYSTEM

(75) Inventors: Norikatsu Furuta, Kyoto-shi (JP); Tomoyuki Yambe, Sendai-shi (JP); Norihiro Sugita, Sendai-shi (JP); Makoto Yoshizawa, Sendai-shi (JP)

(73) Assignees: NINTENDO CO., LTD., Kyoto (JP); TOHOKU UNIVERSITY, Sendai-shi (JP)

(21) Appl. No.: 12/760,872

(22) Filed: Apr. 15, 2010

(30) Foreign Application Priority Data
Jan. 27, 2010 (JP) 2010-016033

(51) Int. Cl. A61B 5/00

(52) U.S. Cl. 600/300

(57) ABSTRACT

A storage medium is provided having stored therein an information processing program executable by a computer of an information processing device which performs predetermined processes based on biological parameters acquired from a user. The information processing program causes the computer to function as: first biological parameter acquisition means for acquiring a first biological parameter from the user during a first period; second biological parameter acquisition means for acquiring a second biological parameter from the user, within the first period, during the acquisition of the first biological parameter, or on and after a first time point after the acquisition of the first biological parameter; and first process execution means for executing a first process from the first time point, based on the first biological parameter and the second biological parameter.

### CROSS REFERENCE TO RELATED APPLICATION

The disclosure of Japanese Patent Application No. 2010-016033, filed on Jan. 27, 2010, is incorporated herein by reference.

### BACKGROUND OF THE INVENTION

1. Field of the Invention

The present invention relates to a storage medium having stored therein an information processing program, an information processing device, an information processing method, and an information processing system. More particularly, the present invention relates to a storage medium having stored therein an information processing program, an information processing device, an information processing method, and an information processing system, which perform predetermined processes based on a biological signal of a user.

2. Description of the Background Art

Takayuki HASEGAWA and Kiyoko YOKOYAMA, “The Relaxation Biofeedback System With Computer and Heart Rate Variability Interaction”, IEICE technical report, ME and bio cybernetics, Institute of Electronics, Information and Communication Engineers, Vol. 103, No. 470, pp. 35-38, Nov. 20, 2003 (hereinafter referred to as Non-Patent Document 1) proposes a biofeedback system for stress management or relaxation treatment. This system estimates and presents a relaxation level in real time, based on measured heart rate information of the user. Moreover, the system interacts with the user to improve a relaxation effect on the user, thereby providing a function of performing biofeedback in a manner adapted for the individual user.

In the biofeedback system described in Non-Patent Document 1, a value called a relaxation level, which is calculated based on the heart rate information or the like of the user, is reflected on and represented by a motion of a character or an environment in a miniscape. The biofeedback system thereby interacts with the user in a manner which improves the relaxation effect. However, this representation only visually presents the relaxation level to the user, so that the user can only recognize a current relaxation level.

Since it is necessary to obtain a value of a relaxation level which is sufficient to be reflected on representation of such as a motion of a character or an environment in a miniscape, a biological signal must be continuously acquired from the user of the system over a long period of time. Accordingly, in the process of acquiring a biological signal from the user in this system, the user faces the system while being strongly aware of being subjected to the process. Moreover, the system cannot perform information processing including presentation adapted for each of the individual characteristics of users reacting in different ways against a variety of daily environments.

### SUMMARY OF THE INVENTION

Therefore, an object of the present invention is to provide a storage medium having stored therein an information processing program relating to biological information, an information processing device, an information processing method, and an information processing system, which are capable of providing information processing including presentation adapted for each of the individual characteristics of users.

The present invention has the following features to attain the object mentioned above.

In one aspect of the present invention, a storage medium is provided having stored therein an information processing program executable by a computer of an information processing device which performs predetermined processes based on biological parameters acquired from a user. This information processing program causes the computer to function as first biological parameter acquisition means, second biological parameter acquisition means, and first process execution means. The first biological parameter acquisition means acquires a first biological parameter from the user during a first period. The second biological parameter acquisition means acquires a second biological parameter from the user, within the first period, during the acquisition of the first biological parameter, or on and after a first time point after the acquisition of the first biological parameter. The first process execution means executes a first process from the first time point, based on the first biological parameter and the second biological parameter. The first biological parameter and the second biological parameter may be of different types or of the same type, depending on the process to be performed. The first biological parameter and the second biological parameter may be parameters representing the same feature (e.g., heat rate).

In one embodiment, the information processing program causes the computer to further function as biological signal acquisition means for acquiring a biological signal from the user. The first biological parameter acquisition means acquires the first biological parameter based on the biological signal acquired by the biological signal acquisition means. Further, the second biological parameter acquisition means acquires the second biological parameter based on the biological signal acquired by the biological signal acquisition means.

In another embodiment, the biological signal acquisition means acquires the biological signal from the user during a second period including the first period.

In still another embodiment, the biological signal acquisition means acquires, as the biological signal, a signal relating to pulse or heartbeat of the user.

In yet another embodiment, the biological signal acquisition means acquires, as the biological signal, at least one selected from the group consisting of a pulse wave, a heart rate, an activity level of sympathetic nervous system, an activity level of parasympathetic nervous system, a coefficient of variance of R-R interval, a cardiac cycle, a respiration frequency, and a pulse wave amplitude of the user.

In another embodiment, the first process execution means executes the first process from the first time point, based on the first biological parameter and the second biological parameter which are acquired during a period corresponding to at least part of a period before the first time point.

In still another embodiment, the first process execution means executes the first process, based on the first biological parameter and the second biological parameter which are based on the biological signal acquired during a period between a time point at which the acquisition of the biological signal is started by the biological signal acquisition means, and the first time point.

In yet another embodiment, the information processing program causes the computer to further function as menu selection means for causing the user to select a desired process from the group consisting of one or more processes including the first process. The first time point is a point at which the user selects a desired process in the menu selection means. The menu selection means causes the user to select a desired process by the first time point.

In another embodiment, the menu selection means causes the user to select a desired process during the first period.

In the above-described information processing program, the first process execution means includes first presentation means for presenting a result of the execution of the first process.

Further, in the above-described information processing program, the first time point is a predetermined point during the acquisition of the first biological parameter. Further, the first presentation means presents the result of the execution of the first process, based on the first biological parameter which is acquired when the first presentation means presents the result of the execution of the first process.

Further, the above-described information processing program causes the computer to further function as login means and logout means. The login means performs a login process which causes the user to log in. On the other hand, the logout means performs a logout process which causes the user to log out, after the login process by the login means. The first biological parameter acquisition means acquires the first biological parameter from the user, during a period corresponding to at least part of a period between the login process by the login means and the logout process by the logout means.

In another embodiment, the first biological parameter acquisition means acquires the first biological parameter from the user at predetermined intervals.

In another embodiment, the first biological parameter acquisition means does not acquire the first biological parameter during a period in which the first process execution means executes the first process.

In another embodiment, the first process execution means executes the first process, based on the first biological parameter which has been acquired by the first biological parameter acquisition means before the execution of the first process.

In another embodiment, the first process execution means executes the first process, based on a predetermined number of the first biological parameters which are acquired by the first biological parameter acquisition means.

Further, the above-described information processing program causes the computer to further function as history storage means. The history storage means stores first biological information in association with the user. The first biological information includes the first biological parameter acquired by the first biological parameter acquisition means, or the second biological parameter acquired by the second biological parameter acquisition means, or a combination of them.

Here, the first process execution means executes the first process also based on the first biological information.

The above-described information processing program causes the computer to further function as second process execution means. The second process execution means executes a second process, based on the first biological information, after the first process.

The above-described information processing program causes the computer to further function as motion/posture information acquisition means. The motion/posture information acquisition means acquires information about a motion and/or posture of the user, from detection means for detecting this information. The first process execution means executes the first process also based on the information acquired by the motion/posture information acquisition means.

Here, the first process execution means prompts the user to take a predetermined motion and/or posture.

In the above-described information processing program, the first process execution means further performs a process for changing the manner of presentation to the user, depending on the first biological parameter, the second biological parameter, and the periods in which the respective biological parameters are acquired.

Further, the above-described information processing program may be used in an information processing device which executes the program, or in an information processing system which includes a plurality of devices configured to be communicable with each other, and performs information processing based on a biological signal acquired from a user.

Further, in an embodiment, the present invention may be provided as an information processing method for performing predetermined processes based on a biological parameter acquired from the user. For example, the information processing method includes a step of executing a first process from a first time point, based on a first biological parameter acquired from the user during a first period, and a second biological parameter which is acquired from the user, within the first period, during the acquisition of the first biological parameter, or on and after a first time point after the acquisition of the first biological parameter.

Note that, as used herein, “biological signal” means an arbitrary amount relating to a biological body, which varies with time or space.

Further, as used herein, “biological parameter” means information to be used for determination relating to a feature or state of a biological body. As long as a biological signal itself acquired from a biological body by using measurement means is used directly for determination relating to a feature or state of the biological body, the biological signal itself can be a biological parameter. Examples of the biological parameter may include, but are not limited to, a heart rate, the number of pulses, an activity level of sympathetic nervous system, an activity level of parasympathetic nervous system, a coefficient of variance of R-R interval, a cardiac cycle, a respiration frequency, an average respiration frequency, a pulse wave, and a pulse wave amplitude.

Further, as used herein, “biological information” means information relating to a biological body, and includes a biological parameter. Examples of the biological information may include, but are not limited to, pulse wave information.

According to the present invention, it is possible to provide an information processing program relating to biological information, an information processing device, an information processing method, and an information processing system, which are capable of providing presentation adapted for each of the individual characteristics of users reacting in different ways against a variety of daily environments.

These and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is an external view showing an example of a game system 1 according to an embodiment of the present invention;

FIG. 2 is a block diagram showing an example of a game apparatus body 5 of FIG. 1;

FIG. 3 is an isometric view of a core unit 70 of FIG. 1 seen from a top rear side thereof;

FIG. 4 is an isometric view of the core unit 70 of FIG. 3 seen from a bottom front side thereof;

FIG. 5 is an isometric view showing a state where an upper casing of the core unit 70 of FIG. 3 is removed;

FIG. 6 is an isometric view showing a state where a lower casing of the core unit 70 of FIG. 4 is removed;

FIG. 7 is a block diagram showing an example of a configuration of the core unit 70 of FIG. 3;

FIG. 8 is a block diagram showing an example of a configuration of a vital sensor 76;

FIG. 9 is a diagram showing an example of pulse wave information which is an example of biological information outputted from the vital sensor 76;

FIG. 10A is a schematic diagram showing a chronological process in which an information processing apparatus for executing a biological information processing program obtains a biological signal from a user, and retains and utilizes the same;

FIG. 10B is a schematic diagram showing a chronological process of executing the biological information processing program;

FIG. 11 is a diagram showing an example of a series of images displayed on a monitor 2;

FIG. 12 is a diagram showing an example of a series of images displayed on the monitor 2;

FIG. 13 is a diagram showing an example of a series of images displayed on the monitor 2;

FIG. 14 is a diagram showing an example of a series of images displayed on the monitor 2;

FIG. 15 is a diagram showing an example of a series of images displayed on the monitor 2;

FIG. 16 is a schematic diagram showing a chronological process of executing the biological information processing program;

FIG. 17 is a diagram showing an example of a series of images displayed on the monitor 2;

FIG. 18 is a diagram showing an example of a series of images displayed on the monitor 2;

FIG. 19 is a diagram showing an example of a series of images displayed on the monitor 2;

FIG. 20 is a diagram showing an example of a series of images displayed on the monitor 2;

FIG. 21 is a diagram showing an example of a series of images displayed on the monitor 2;

FIG. 22 is a diagram showing an example of a series of images displayed on the monitor 2;

FIG. 23 is a diagram showing an example of a series of images displayed on the monitor 2;

FIG. 24 is a diagram showing an example of a series of images displayed on the monitor 2;

FIG. 25 is a diagram showing an example of a series of images displayed on the monitor 2;

FIG. 26 is a diagram showing an example of a series of images displayed on the monitor 2;

FIG. 27 is a diagram showing an example of a series of images displayed on the monitor 2;

FIG. 28A is a diagram showing an example of main data and programs stored in a main memory of the game apparatus body 5;

FIG. 28B is a diagram (which follows FIG. 28A) showing an example of main data and programs stored in a main memory of the game apparatus body 5;

FIG. 28C is a diagram (which follows FIG. 28B) showing an example of main data and programs stored in a main memory of the game apparatus body 5;

FIG. 29 is a flowchart showing an example of an information processing executed in the game apparatus body 5;

FIG. 30 is a flowchart showing an example of a subroutine process in the flowchart of FIG. 29;

FIG. 31 is a flowchart showing an example of a subroutine process in the flowchart of FIG. 29;

FIG. 32A is a flowchart showing a process of an example of a game application utilizing biological information;

FIG. 32B is a flowchart (which follows FIG. 32A) showing a process of an example of a game application utilizing biological information; and

FIG. 33 is a flowchart showing a process of an example of a game application utilizing biological information.

### DESCRIPTION OF THE PREFERRED EMBODIMENTS

(Fundamental Structure of Information Processing System)

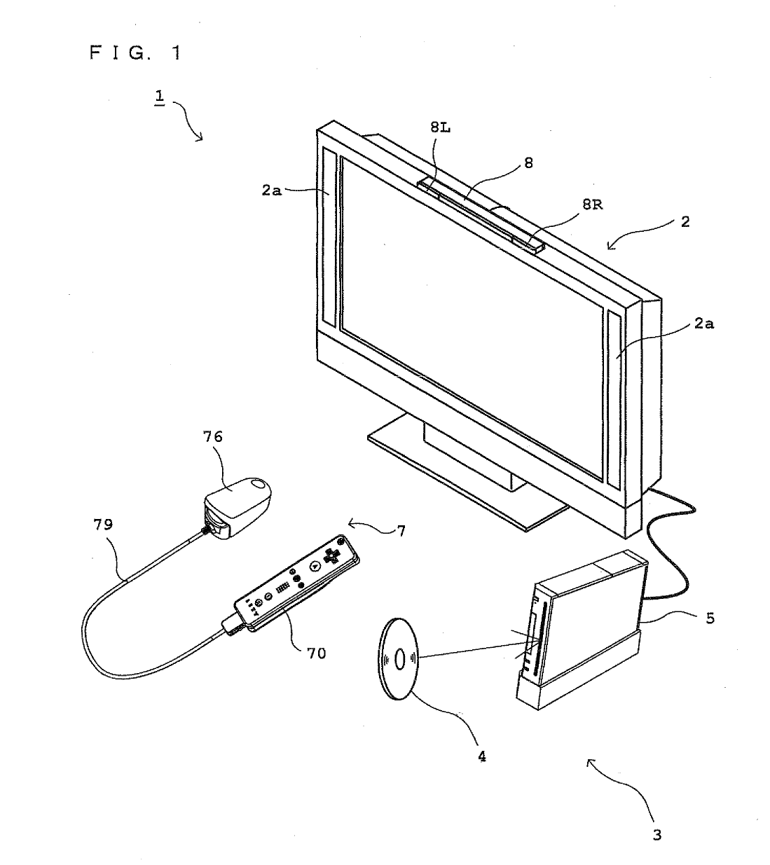

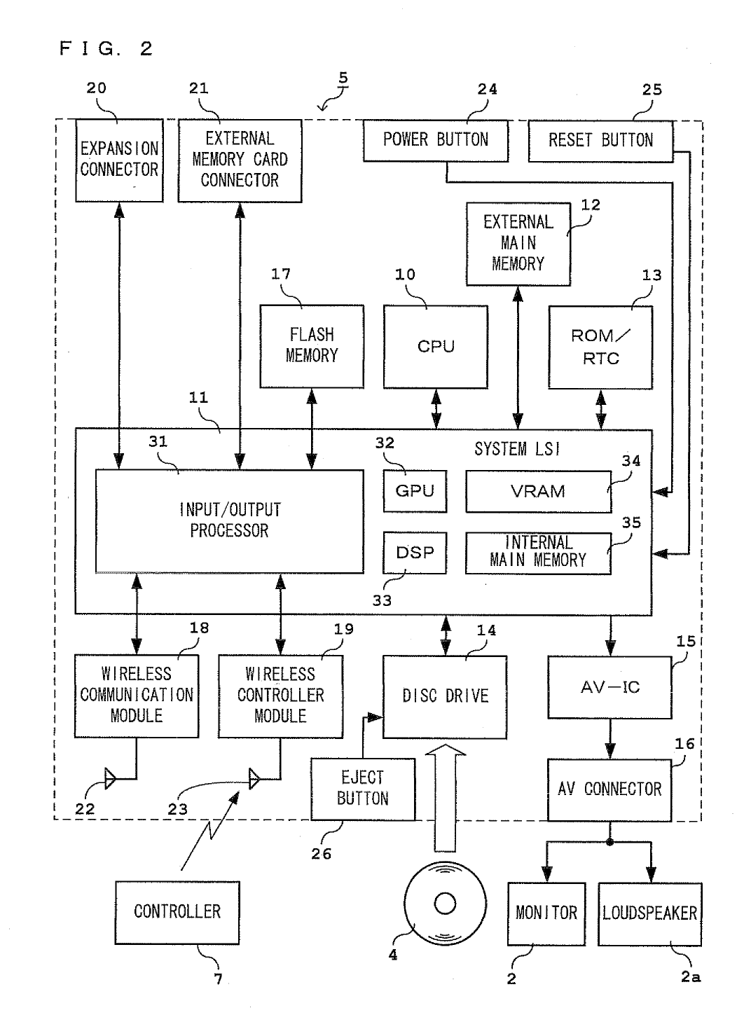

The following will describe, with reference to FIG. 1, an example of an information processing system for executing an information processing program according to the present invention. Hereinafter, in order to give a specific description, a description will be given using, as the information processing system, a game system 1 including a stationary game apparatus body 5. FIG. 1 is an external view showing an example of a game system 1 including a stationary game apparatus 3. FIG. 2 is a block diagram showing an example of the game apparatus body 5. The game system 1 will be described below.

As shown in FIG. 1, the game system 1 includes: a home-use TV receiver 2 (hereinafter, referred to as a monitor 2) which is an example of display means; and the stationary game apparatus 3 connected to the monitor 2 via a connection cord. The monitor 2 has loudspeakers 2a for outputting, in the form of sound, an audio signal outputted from the game apparatus 3. The game apparatus 3 includes: an optical disc 4 storing a biological information processing program that is an example of an information processing program of the present invention; the game apparatus body 5 having a computer for executing the biological information processing program of the optical disc 4 to cause the monitor 2 to output and display a game screen; and a controller 7 for providing the game apparatus body 5 with necessary operation information for a game in which a character or the like displayed in the game screen is controlled.

The game apparatus body 5 has a wireless controller module 19 therein (refer to FIG. 2). The wireless controller module 19 receives data wirelessly transmitted from the controller 7, and transmits data from the game apparatus body 5 to the controller 7. In this manner, the controller 7 and the game apparatus body 5 are connected by wireless communication. Further, the optical disc 4 as an example of an exchangeable information storage medium is detachably mounted on the game apparatus body 5.

On the game apparatus body 5, a flash memory 17 (refer to FIG. 2) is mounted, the flash memory 17 acting as a backup memory for fixedly storing such data as saved data. The game apparatus body 5 executes the biological information processing program or the like stored in the optical disc 4, and displays a result thereof as a game image on the monitor 2. The biological information processing program or the like to be executed may be previously stored not only in the optical disc 4, but also in the flash memory 17. The game apparatus body 5 can reproduce a state of the game played in the past, by using the saved data stored in the flash memory 17, and display a game image of the reproduced state on the monitor 2. A user of the game apparatus 3 can enjoy advancing in the game by operating the controller 7 while watching the game image displayed on the monitor 2.

By using the technology of, for example, Bluetooth (registered trademark), the controller 7 wirelessly transmits transmission data, such as operation information and biological information, to the game apparatus body 5 having the wireless controller module 19 therein.

The controller 7 includes a core unit 70 and a vital sensor 76. The core unit 70 and the vital sensor 76 are connected to each other via a flexible connection cable 79. The core unit 70 is operation means mainly for controlling an object or the like displayed on a display screen of the monitor 2. The vital sensor 76 is attached to the user's body (e.g., to the user's finger). The vital sensor obtains biological signals from the user, and sends biological information to the core unit 70 via the connection cable 79.

The core unit 70 includes a housing, which is small enough to be held by one hand, and a plurality of operation buttons (including a cross key, a stick or the like) exposed at a surface of the housing. As described later in detail, the core unit 70 includes an imaging information calculation section 74 for taking an image of a view seen from the core unit 70. As an example of imaging targets of the imaging information calculation section 74, two LED modules 81, and 82 (hereinafter, referred to as “markers 8L and 8R”) are provided in the vicinity of the display screen of the monitor 2. These markers 8L and 8R each output, for example, infrared light forward from the monitor 2.

The controller 7 (e.g., the core unit 70) is capable of receiving, via a communication section 75, transmission data wirelessly transmitted from the wireless controller module 19 of the game apparatus body 5, and generating a sound or vibration based on the transmission data.

In this example, the core unit 70 and the vital sensor 76 are connected by the flexible connection cable 79. However, the connection cable 79 can be eliminated by mounting a wireless unit on the vital sensor 76. For example, by mounting a Bluetooth (registered trademark) unit on the vital sensor 76 as a wireless unit, transmission of biological information data from the vital sensor 76 to the core unit 70 or to the game apparatus body 5 is enabled. Further, the core unit 70 and the vital sensor 76 may be integrated, by fixedly providing the vital sensor 76 on the core unit 70. In this case, a user can use the vital sensor 76 integrated with the core unit 70.

(Internal Structure of Game Apparatus Body)

The following will describe an internal configuration of the game apparatus body 5, with reference to FIG. 2. FIG. 2 is a block diagram showing the internal structure of the game apparatus body 5. The game apparatus body 5 has a CPU (Central Processing Unit) 10, a system LEI (Large Scale Integration) 11, an external main memory 12, a ROM/RTC (Read Only Memory/Real Time Clock) 13, a disc drive 14, an AV-IC (Audio Video-Integrated Circuit) 15, and the like.

The CPU 10 performs biological information processing by executing the biological information processing program stored in the optical disc 4, and acts as a biological information processor. When a game application is included in the biological information processing program, the CPU 10 executes game processing by executing the game application stored in the optical disc 4, and acts as a game processor.

The CPU 10 is connected to the system LSI 11. In addition to the CPU 10, the external main memory 12, the ROM/RTC 13, the disc drive 14 and the AV-IC 15 are connected to the system LSI 11. The system LSI 11 performs processing such as: controlling data transfer among the components connected to the system LSI 11; generating an image to be displayed; obtaining data from external devices; and the like. An internal structure of the system LSI 11 will be described later. The external main memory 12 that is a volatile memory stores a program, for example, a biological information processing program loaded from the optical disc 4, or a biological information processing program loaded from the flash memory 17, and also stores various data. The external main memory 12 is used as a work area or buffer area of the CPU 10. The ROM/RTC 13 has a ROM in which a boot program for the game apparatus body 5 is incorporated (so-called a boot ROM), and has a clock circuit (RTC) which counts the time. The disc drive 14 reads program data, texture data and the like from the optical disc 4, and writes the read data into a later-described internal main memory 35 or into the external main memory 12.

On the system LSI 11, an input/output processor 31, a GPU (Graphic Processor Unit) 32, a DSP (Digital Signal Processor) 33, a VRAM (Video RAM) 34, and the internal main memory 35 are provided. Although not shown, these components 31 to 35 are connected to each other via an internal bus.

The GPU 32 is a part of rendering means, and generates an image in accordance with a graphics command from the CPU 10. The VRAM 34 stores necessary data for the GPO 32 to execute the graphics command (data such as polygon data, texture data and the like). At the time of generating the image, the GPU 32 uses the data stored in the VRAM 34, thereby generating image data.

The DSP 33 acts as an audio processor, and generates audio data by using sound data and sound waveform (tone) data stored in the internal main memory 35 and in the external main memory 12.

The image data and the audio data generated in the above manner are read by the AV-IC 15. The AV-IC 15 outputs the read image data to the monitor 2 via the AV connector 16, and outputs the read audio data to the loudspeakers 2a embedded in the monitor 2. As a result, an image is displayed on the monitor 2 and a sound is outputted from the loudspeakers 2a.

The input/output processor (I/O processor) 31 performs, for example, data transmission/reception to/from components connected thereto, and data downloading from external devices. The input/output processor 31 is connected to the flash memory 17, a wireless communication module 18, the wireless controller module 19, an expansion connector 20, and an external memory card connector 21. An antenna 22 is connected to the wireless communication module 18, and an antenna 23 is connected to the wireless controller module 19.

The input/output processor 31 is connected to a network via the wireless communication module 18 and the antenna 22 so as to be able to communicate with other game apparatuses and various servers connected to the network. The input/output processor 31 regularly accesses the flash memory 17 to detect presence or absence of data that is required to be transmitted to the network. If such data is present, the data is transmitted to the network via the wireless communication module 18 and the antenna 22. Also, the input/output processor 31 receives, via the network, the antenna 22 and the wireless communication module 18, data transmitted from other game apparatuses or data downloaded from a download server, and stores the received data in the flash memory 17. By executing the biological information processing program, the CPU 10 reads the data stored in the flash memory 17, and the biological information processing program uses the read data. In addition to the data transmitted and received between the game apparatus body 5 and other game apparatuses or various servers, the flash memory 17 may store saved data of a game that is played using the game apparatus body 5 (such as result data or progress data of the game).

Further, the input/output processor 31 receives, via the antenna 23 and the wireless controller module 19, operation data or the like transmitted from the controller 7, and stores (temporarily) the operation data or the like in a buffer area of the internal main memory 35 or of the external main memory 12. Note that, similarly to the external main memory 12, the internal main memory 35 may store a program, for example, a biological information processing program loaded from the optical disc 4 or a biological information processing program loaded from the flash memory 17, and also store various data. The internal main memory 35 may be used as a work area or buffer area of the CPU 10.

In addition, the expansion connector 20 and the external memory card connector 21 are connected to the input/output processor 31. The expansion connector 20 is a connector for such interface as USB, SCSI or the like. The expansion connector 20, instead of the wireless communication module 18, is able to perform communication with the network by being connected to such a medium as an external storage medium, to such a peripheral device as another controller, or to a connector for wired communication. The external memory card connector 21 is a connector to be connected to an external storage medium such as a memory card. For example, the input/output processor 31 is able to access the external storage medium via the expansion connector 20 or the external memory card connector 21 to store or read data in or from the external storage medium.

On the game apparatus body 5 (e.g., on a front main surface thereof), a power button 24 of the game apparatus body 5, a reset button 25 for resetting game processing, an insertion slot for mounting the optical disc 4 in a detachable manner, an eject button 26 for ejecting the optical disc 4 from the insertion slot of the game apparatus body 5, and the like are provided. The power button 24 and the reset button 25 are connected to the system LSI 11. When the power button 24 is turned on, each component of the game apparatus body 5 is supplied with power via an AC adaptor that is not shown. When the reset button 25 is pressed, the system LSI 11 re-executes the boot program of the game apparatus body 5. The eject button 26 is connected to the disc drive 14. When the eject button 26 is pressed, the optical disc 4 is ejected from the disc drive 14.

(Fundamental Structure of Core Unit)

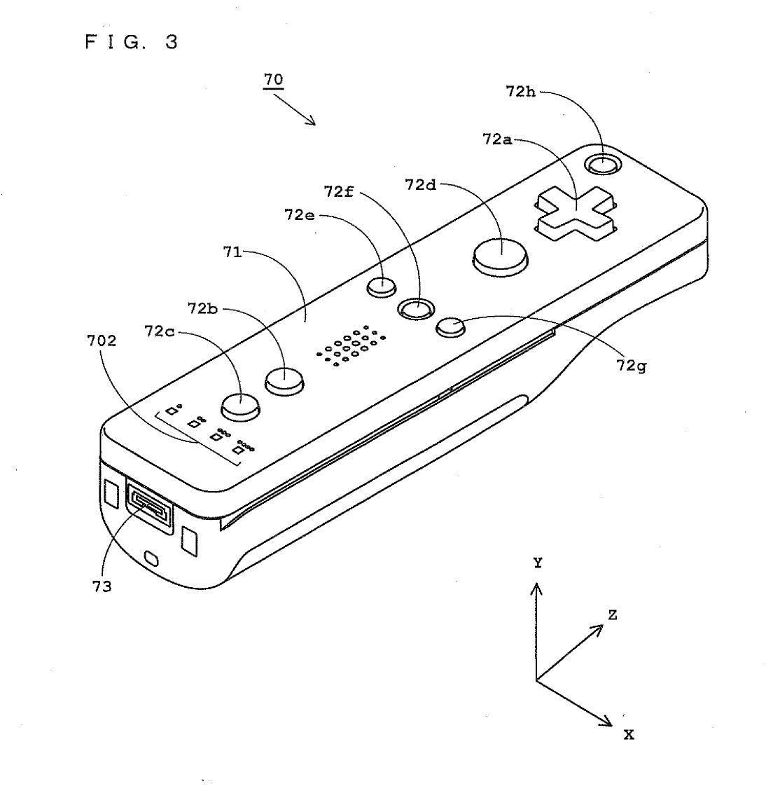

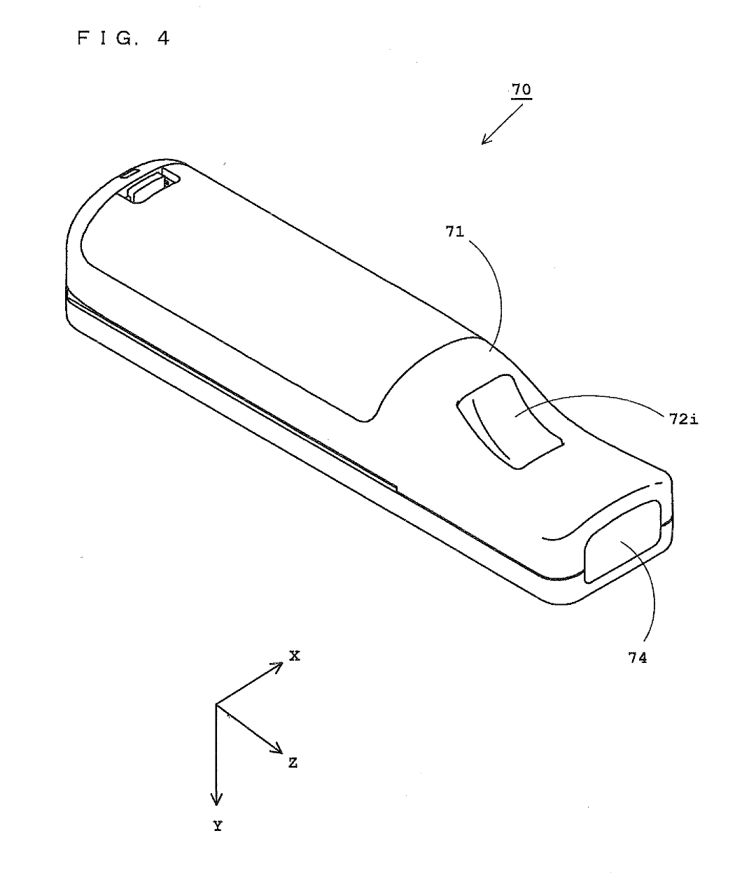

With reference to FIGS. 3 and 4, the core unit 70 will be described. FIG. 3 is an isometric view of the core unit 70 seen from a top rear side thereof. FIG. 4 is an isometric view of the core unit 70 seen from a bottom front side thereof.

As shown in FIGS. 3 and 4, the core unit 70 includes a housing 71 formed by plastic molding or the like. The housing 71 has a plurality of operation sections 72 provided thereon. The housing 71 has an approximately parallelepiped shape extending in a longitudinal direction from front to rear. The overall size of the housing 71 is small enough to be held by one hand of an adult or even a child.

At the center of a front part of a top surface of the housing 71, a cross key 72a is provided. The cross key 72a is a cross-shaped four-direction push switch. The cross key 72a includes operation portions corresponding to four directions (front, rear, right and left), which are respectively located on cross-shaped projecting portions arranged at intervals of 90 degrees. A user selects one of the front, rear, right and left directions by pressing one of the operation portions of the cross key 72a. Through an operation of the cross key 72a, the user can, for example, designate a direction in which a player character or the like appearing in a virtual game world is to move, or give an instruction to select one of a plurality of options.

The cross key 72a is an operation section for outputting an operation signal in accordance with the aforementioned direction input operation performed by the user. Such an operation section may be provided in a different form. For example, an operation section, which has four push switches arranged in a cross formation and which is capable of outputting an operation signal in accordance with pressing of one of the push switches by the user, may be provided. Alternatively, an operation section, which has a composite switch having, in addition to the above four push switches, a center switch provided at an intersection point of the above cross formation, may be provided. Still alternatively, the cross key 72a may be replaced with an operation section which includes an inclinable stick (so-called a joy stick) projecting from the top surface of the housing 71 and which outputs an operation signal in accordance with an inclining direction of the stick. Still alternatively, the cross key 72a may be replaced with an operation section which includes a horizontally-slidable disc-shaped member and which outputs an operation signal in accordance with a sliding direction of the disc-shaped member. Still alternatively, the cross key 72a may be replaced with a touch pad.

Behind the cross key 72a on the top surface of the housing 71, a plurality of operation buttons 72b to 72g are provided. The operation buttons 72b to 72g are each an operation section for, when the user presses a head thereof, outputting a corresponding operation signal. For example, functions as a 1st button, a 2nd button and an A button are assigned to the operation buttons 72b to 72d. Also, functions as a minus button, a home button and a plus button are assigned to the operation buttons 72e to 72g. The operation functions are assigned to the respective operation buttons 72a to 72g in accordance with the biological information processing program executed by the game apparatus body 5. In the exemplary arrangement shown in FIG. 3, the operation buttons 72b to 72d are arranged in a line at the center on the top surface of the housing 71 in a front-rear direction. The operation buttons 72e to 72g are arranged on the top surface of the housing 71 in a line in a left-right direction between the operation buttons 72b and 72d. The operation button 72f has a top surface thereof buried in the top surface of the housing 71, so as not to be inadvertently pressed by the user.

In front of the cross key 72a on the top surface of the housing 71, an operation button 72h is provided. The operation button 72h is a power switch for turning on and off the game apparatus body 5 by remote control. The operation button 72h also has a top surface buried in the top surface of the housing 71, so as not to be inadvertently pressed by the user.

Behind the operation button 72c on the top surface of the housing 71, a plurality of LEDs 702 are provided. Here, a controller type (a number) is assigned to the core unit 70 such that the core unit 70 is distinguishable from other controllers. The LEDs 702 are used for, e.g., informing the user of the controller type currently set for the core unit 70. Specifically, a signal is transmitted from the wireless controller module 19 to the core unit 70 such that one of the plurality of LEDs 702, which corresponds to the controller type of the core unit 70, is lit up.

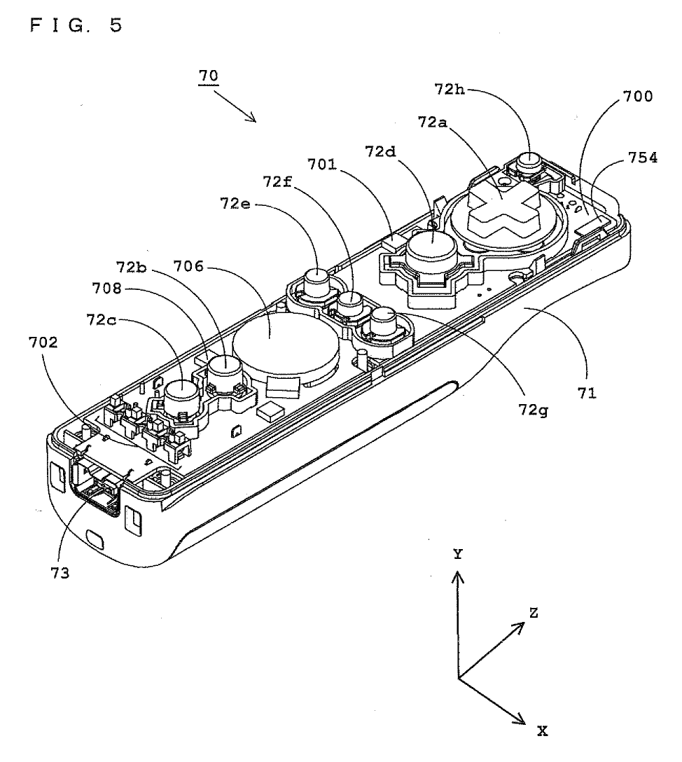

On the top surface of the housing 71, sound holes for outputting sounds from a later-described speaker (a speaker 706 shown in FIG. 5) to the external space are formed between the operation button 72b and the operation buttons 72e to 72g.

On the bottom surface of the housing 71, a recessed portion is formed. The recessed portion on the bottom surface of the housing 71 is formed in a position in which an index finger or middle finger of the user is located when the user holds the core unit 70 with one hand so as to point a front surface thereof to the markers BL and BR. On a slope surface of the recessed portion, an operation button 72i is provided. The operation button 72i is an operation section acting as, for example, a B button.

On the front surface of the housing 71, an image pickup element 743 that is a part of the imaging information calculation section 74 is provided. The imaging information calculation section 74 is a system for: analyzing image data of an image taken by the core unit 70; identifying an area having a high brightness in the image; and detecting a position of the center of gravity, the size, and the like of the area. The imaging information calculation section 74 has, for example, a maximum sampling period of approximately 200 frames/sec, and therefore can trace and analyze even a relatively fast motion of the core unit 70. The configuration of the imaging information calculation section 74 will be described later in detail. On the rear surface of the housing 71, a connector 73 is provided. The connector 73 is, for example, an edge connector, and is used for engaging and connecting the core unit 70 with a connection cable, for example.

In order to give a specific description below, a coordinate system set with respect to the core unit 70 will be defined. As shown in FIGS. 3 and 4, an X-axis, a Y-axis and a Z-axis, which are perpendicular to one another, are defined with respect to the core unit 70. Specifically, the longitudinal direction of the housing 71, which is the front-rear direction of the core unit 70, is defined as the Z-axis, and a direction along the Z-axis toward the front surface (a surface on which the imaging information calculation section 74 is provided) of the core unit 70 is a Z-axis positive direction. The up-down direction of the core unit 70 is defined as the Y-axis, and a direction along the Y-axis toward the top surface (a surface on which the operation button 72a is provided) of the housing 71 is defined as a Y-axis positive direction. The left-right direction of the core unit 70 is defined as the X-axis, and a direction along the X-axis toward the right side surface (a side surface shown in FIG. 3) of the housing 71 is defined as an X-axis positive direction.

### (Internal Structure of Core Unit)

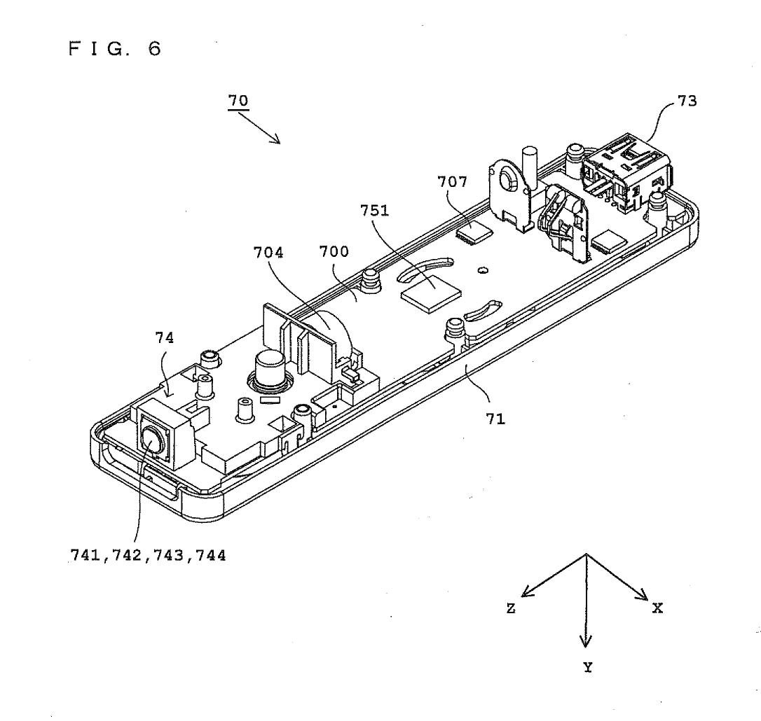

Next, an internal structure of the core unit 70 will be described with reference to FIGS. 5 and 6. FIG. 5 is an isometric view, seen from a rear surface side of the core unit 70, showing that an upper casing (a part of the housing 71) of the core unit 70 is removed. FIG. 6 is an isometric view, seen from a front surface side of the core unit 70, showing that a lower casing (a part of the housing 71) of the core unit 70 is removed. Here, FIG. 6 is an isometric view showing a reverse side of a substrate 700 shown in FIG. 5.

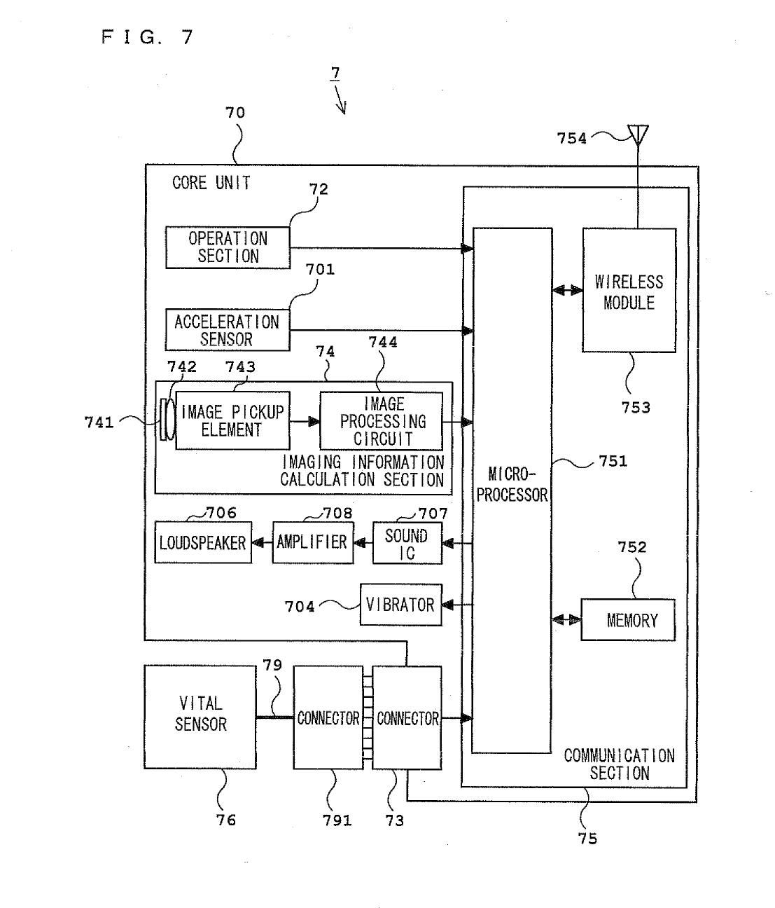

As shown in FIG. 5, the substrate 700 is fixedly provided inside the housing 71. On a top main surface of the substrate 700, the operation buttons 72a to 72h, an acceleration sensor 701, the LEDs 702, an antenna 754 and the like are provided. These elements are connected to, for example, a microcomputer 751 (refer to FIGS. 6 and 7) by wiring (not shown) formed on the substrate 700 and the like. A wireless module 753 (refer to FIG. 7) and the antenna 754 allow the core unit 70 to act as a wireless controller. Inside the housing 71, a quartz oscillator, which is not shown, is provided, and the quartz oscillator generates a reference clock of the later-described microcomputer 751. Further, the speaker 706 and an amplifier 708 are provided on the top main surface of the substrate 700. The acceleration sensor 701 is provided, on the substrate 700, to the left side of the operation button 72d (i.e., provided not on a central part but on a peripheral part of the substrate 700). For this reason, in response to the core unit 70 having rotated around an axis of the longitudinal direction of the core unit 70, the acceleration sensor 701 is able to detect, in addition to a change in a direction of the gravitational acceleration, acceleration containing a centrifugal component, and the game apparatus body 5 or the like is able to determine, based on detected acceleration data, a motion of the core unit 70 by predetermined calculation with favorable sensitivity.

As shown in FIG. 6, at a front edge of the bottom main surface of the substrate 700, the imaging information calculation section 74 is provided. The imaging information calculation section 74 includes an infrared filter 741, a lens 742, the image pickup element 743, and an image processing circuit 744, which are located in said order from the front surface of the core unit 70. These elements are attached to the bottom main surface of the substrate 700. At a rear edge of the bottom main surface of the substrate 700, the connector 73 is attached. Further, a sound IC 707 and the microcomputer 751 are provided on the bottom main surface of the substrate 700. The sound IC 707 is connected to the microcomputer 751 and the amplifier 708 by wiring formed on the substrate 700 and the like, and outputs an audio signal via the amplifier 708 to the speaker 706 in response to sound data transmitted from the game apparatus body 5.

On the bottom main surface of the substrate 700, a vibrator 704 is attached. The vibrator 704 may be, for example, a vibration motor or a solenoid. The vibrator 704 is connected to the microcomputer 751 by wiring formed on the substrate 700 and the like, and is activated or deactivated in accordance with vibration data transmitted from the game apparatus body 5. The core unit 70 is vibrated by actuation of the vibrator 704, and the vibration is conveyed to the user's hand holding the core unit 70. Thus, a so-called vibration-feedback game is realized. Since the vibrator 704 is provided at a relatively forward position in the housing 71, the housing 71 held by the user significantly vibrates, and allows the user to easily feel the vibration.

(Internal Structure of Controller)

Next, an internal structure of the controller 7 will be described with reference to FIG. 7. FIG. 7 is a block diagram showing an example of the internal structure of the controller 7.

As shown in FIG. 7, the core unit 70 includes the communication section 75 in addition to the above-described operation sections 72, the imaging information calculation section 74, the acceleration sensor 701, the vibrator 704, the speaker 706, the sound IC 707, and the amplifier 708. The vital sensor 76 is connected to the microcomputer 751 via the connection cable 79 and connectors 791 and 73.

The imaging Information calculation section 74 includes the infrared filter 741, the lens 742, the image pickup element 743, and the image processing circuit 744. The infrared filter 741 allows, among lights incident thereon through the front surface of the core unit 70, only infrared light to pass therethrough. The lens 742 condenses the infrared light having passed through the infrared filter 741, and outputs the condensed infrared light to the image pickup element 743. The image pickup element 743 is a solid-state image pickup element such as a CMOS sensor, CCD or the like. The image pickup element 743 takes an image of the infrared light condensed by the lens 742. In other words, the image pickup element 743 takes an image of only the infrared light having passed through the infrared filter 741. Then, the image pickup element 743 generates image data of the image. The image data generated by the image pickup element 743 is processed by the image processing circuit 744. Specifically, the image processing circuit 744 processes the image data obtained from the image pickup element 743, and detects a high brightness area of the image, and outputs, to the communication section 75, process result data indicating the results of the detection, for example, position coordinates, a square measure of the high brightness area. The imaging information calculation section 74 is fixed to the housing 71 of the core unit 70. The imaging direction of the imaging information calculation section 74 can be changed by changing the facing direction of the housing 71.

Preferably, the core unit 70 includes a triaxial (X-axis, Y-axis, and Z-axis) acceleration sensor 701. The triaxial acceleration sensor 701 detects linear acceleration in three directions, i.e., the up-down direction (the I-axis shown in FIG. 3), the left-right direction (the X-axis shown in FIG. 3), and the front-rear direction (the Z-axis shown in FIG. 3). Alternatively, an accelerometer capable of detecting linear acceleration along at least one axis direction (e.g. Z-axis direction) may be used. As a non-limiting example, the acceleration sensor 701 may be of the type available from Analog Devices, Inc. or STMicroelectronics N.V. Preferably, the acceleration sensor 701 is of an electrostatic capacitance or capacitance-coupling type that is based on silicon micro-machined MEMS (microelectromechanical systems) technology. However, any other suitable accelerometer technology (e.g., piezoelectric type or piezoresistance type) now existing or later developed may be used to provide the acceleration sensor 701.

Accelerometers, as used in the acceleration sensor 701, are only capable of detecting acceleration along a straight line (linear acceleration) corresponding to each axis of the acceleration sensor 701. In other words, the direct output from the acceleration sensor 701 is limited to signals indicative of linear acceleration (static or dynamic) along each of the three axes thereof. As a result, the acceleration sensor 701 cannot directly detect movement along a non-linear (e.g., arcuate) path, rotation, rotational movement, angular displacement, inclination, position, orientation or any other physical characteristic.

However, through processing by a computer such as a processor of the game apparatus (e.g., the CPU 10) or a processor of the controller (e.g., the microcomputer 751) based on the acceleration signals outputted from the acceleration sensor 701, additional information relating to the core unit 70 can be inferred or calculated (determined), as one skilled in the art will readily understand from the description herein.

For example, when the processing is performed by the computer on the assumption that the core unit 70 having the acceleration sensor 701 mounted therein is in a static state (i.e., when the processing is performed assuming that acceleration detected by the acceleration sensor is only the gravitational acceleration), if the core unit 70 is actually in a static state, the detected acceleration is used to determine whether or not the core unit 70 is inclined with respect to the direction of gravity or how many degrees the core unit 70 is inclined with respect to the direction of gravity.

More specifically, when a state where a detection axis of the acceleration sensor 701 extends in a vertically downward direction is set as a standard state, it is possible to determine whether or not the core unit 70 is inclined with respect to the vertically downward direction, based on whether or not 1G (gravitational acceleration) is being applied in a direction along the detection axis of the acceleration sensor 701. It is also possible to determine how many degrees the core unit 70 is inclined with respect to the vertically downward direction, based on the magnitude of acceleration applied in the direction along the detection axis. In addition, in the case where the acceleration sensor 701 is capable of detecting acceleration along multiple axis directions, it is possible to determine in detail how many degrees the core unit 70 is inclined with respect to the direction of gravity, through processing of acceleration signals detected for each axis. In this case, a processor may perform processing, based on an output from the acceleration sensor 701, for calculating data indicating an inclination angle of the core unit 70. Alternatively, processing may be performed so as to infer a rough inclination of the core unit 70 based on the output from the acceleration sensor 701 without performing the processing for calculating data indicating an inclination angle. In this manner, the acceleration sensor 701 can be used in combination with the processor to determine an inclination, orientation or position of the core unit 70.

On the other hand, on the assumption that the acceleration sensor 701 is in a dynamic state, the acceleration sensor 701 detects acceleration corresponding to a movement of the acceleration sensor 701 in addition to a gravitational acceleration component. Thus, it is possible to determine, for example, a direction of the movement of the core unit 70 by eliminating the gravitational acceleration component through predetermined processing.

More specifically, various movements and/or positions of the core unit 70 can be calculated through processing of the acceleration signals generated by the acceleration sensor 701 when the core unit 70 including the acceleration sensor 701 is subjected to dynamic acceleration by the hand of a user. It is noted that even on the assumption that the acceleration sensor 701 is in a dynamic state, it is possible to determine an inclination of the core unit 70 with respect to the direction of gravity, by eliminating acceleration corresponding to a movement of the acceleration sensor 701 through predetermined processing.

In another example, the acceleration sensor 701 may include an embedded signal processor or other type of dedicated processor for performing any desired processing of the acceleration signals outputted from the accelerometers therein prior to outputting signals to the microcomputer 751. For example, the embedded or dedicated processor may convert the detected acceleration signal to a corresponding inclination angle (or into other preferred parameter) when the acceleration sensor 701 is intended to detect static acceleration (e.g., gravitational acceleration). Data indicating the acceleration detected by the acceleration sensor 701 is outputted to the communication section 75.

In still another example, the acceleration sensor 701 may be replaced with a gyro-sensor of any suitable technology incorporating, for example, a rotating or vibrating element. Exemplary MEMS gyro-sensors that may be used in this embodiment are available from Analog Devices, Inc. Unlike the linear acceleration sensor 701, a gyro-sensor is capable of directly detecting rotation (or angular rate) around an axis defined by the gyroscopic element (or elements) therein. Thus, due to the fundamental differences between a gyro-sensor and an acceleration sensor, corresponding changes need to be made to the processing operations that are performed on the output signals from these devices depending on which device is selected for a particular application.

Specifically, when a gyro-sensor is used instead of an acceleration sensor to calculate an inclination and orientation, significant changes are necessary. More specifically, when a gyro-sensor is used, the value of inclination is initialized at the start of detection. Then, angular velocity data which is outputted from the gyro-sensor is integrated. Next, a change amount in inclination from the value of inclination previously initialized is calculated. In this case, the calculated inclination is obtained as a value corresponding to an angle. In contrast, when an acceleration sensor is used to calculate the inclination, the inclination is calculated by comparing the value of the gravitational acceleration of each axial component with a predetermined reference. Therefore, the calculated inclination can be represented as a vector. Thus, without performing initialization, an absolute direction detected using an accelerometer can be obtained. The type of the value calculated as an inclination is also different between a gyro-sensor and an acceleration sensor; i.e., the value is an angle when a gyro-sensor is used and is a vector when an acceleration sensor is used. Therefore, when a gyro-sensor is used instead of an acceleration sensor, data of inclination also needs to be processed by a predetermined conversion that takes into account the fundamental differences between these two devices. Due to the fact that the property of gyroscopes is known to one skilled in the art, as well as the fundamental differences between accelerometers and gyroscopes, further details are not provided herein. While gyro-sensors provide certain advantages due to their ability to directly detect rotation, acceleration sensors are generally more cost-effective as compared with the gyro-sensors when used for the controller of the present embodiment.

The communication section 75 includes the microcomputer 751, a memory 752, the wireless module 753, and the antenna 754. The microcomputer 751 controls the wireless module 753 that wirelessly transmits transmission data, while using the memory 752 as a storage area during processing. The microcomputer 751 also controls operations of the sound IC 707 and the vibrator 704 in accordance with data which is transmitted from the game apparatus body 5 and received by the wireless module 753 via the antenna 754. The sound IC 707 processes sound data or the like which is transmitted from the game apparatus body 5 via the communication section 75. Further, the microcomputer 751 activates the vibrator 704 in accordance with vibration data or the like (e.g., a signal for causing the vibrator 704 to be ON or OFF) which is transmitted from the game apparatus body 5 via the communication section 75.

Operation signals from the operation sections 72 provided on the core unit 70 (key data), acceleration signals from the acceleration sensor 701 with respect to the three axial directions (X-, Y- and Z-axis direction acceleration data), and the process result data from the imaging information calculation section 74, are outputted to the microcomputer 751. Also, biological signals (biological information data) from the vital sensor 76 are outputted to the microcomputer 751 via the connection cable 79.

The microcomputer 751 temporarily stores the inputted data (the key data, the X-, Y- and Z-axis direction acceleration data, the process result data, and the biological information data) in the memory 752 as transmission data to be transmitted to the wireless controller module 19. Here, wireless transmission from the communication section 75 to the wireless controller module 19 is performed at predetermined time intervals. Since game processing is generally performed at a cycle of 1/60 sec, the wireless transmission needs to be performed at a shorter cycle.

Specifically, game processing is performed at a cycle of 16.7 ms ( 1/60 sec), and a transmission interval of the communication section 75 configured using the Bluetooth (registered trademark) technology is 5 ms. When a timing of performing transmission to the wireless controller module 19 arrives, the microcomputer 751 outputs, to the wireless module 753, the transmission data stored in the memory 752 as a series of operation information. Then, using the Bluetooth (registered trademark) technology, the wireless module 753 radiates, from the antenna 754, a radio signal indicating the operation information by using a carrier wave of a predetermined frequency. Thus, the key data from the operation sections 72 provided on the core unit 70, the X-, Y- and Z-axis direction acceleration data from the acceleration sensor 701, the process result data from the imaging information calculation section 74, and the biological information data from the vital sensor 76, are transmitted from the core unit 70. The wireless controller module 19 of the game apparatus body 5 receives the radio signal, and the game apparatus body 5 demodulates or decodes the radio signal to obtain the series of operation information (the key data, the X-, Y- and Z-axis direction acceleration data, the process result data, and the biological information data). In accordance with the series of obtained operation information and the game program, the CPU 10 of the game apparatus body 5 performs game processing. In the case where the communication section 75 is configured using the Bluetooth (registered trademark) technology, the communication section 75 may have a function of receiving transmission data wirelessly transmitted from other devices.

(Fundamental Structure of Vital Sensor)

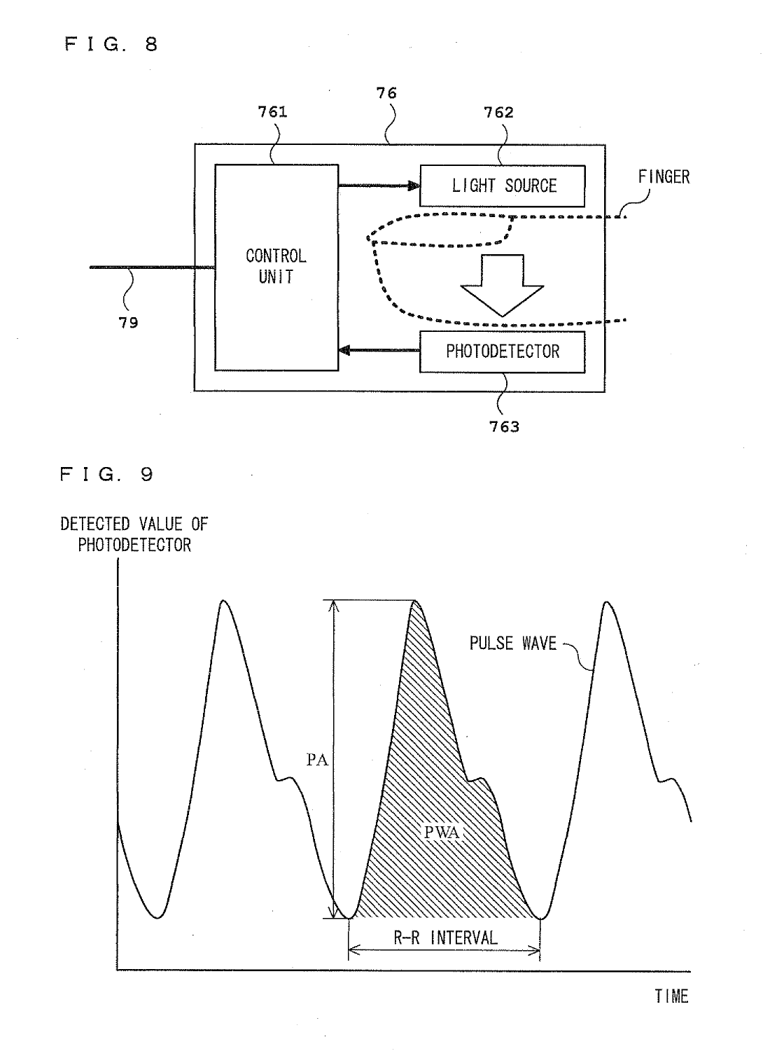

Next, the vital sensor 76 will be described with reference to FIGS. 8 and 9. FIG. 8 is a block diagram showing an example of a structure of the vital sensor 76. FIG. 9 is a diagram showing pulse wave information as an example of biological information outputted from the vital sensor 76.

As shown in FIG. 8, the vital sensor 76 includes a control section 761, a light source 762, and a photodetector 763.

The light source 762 and the photodetector 763 constitute a transmission-type digital-plethysmography sensor, which is an example of a sensor for acquiring a biological signal of the user. The light source 762 includes, for example, an infrared LED which emits infrared light having a predetermined wavelength (e.g., 940 nm) toward the photodetector 763. On the other hand, the photodetector 763 includes, for example, an infrared photoresistor which senses light emitted by the light source 762, depending on the wavelength of the emitted light. The light source 762 and the photodetector 763 are arranged, facing each other, with a predetermined space (cavity) being interposed therebetween.

Here, hemoglobin which exists in human blood absorbs infrared light. For example, a portion (e.g., a fingertip) of the body of the user is inserted in the space between the light source 762 and the photodetector 763. Thereby, infrared light emitted from the light source 762 is partially absorbed by hemoglobin existing in the inserted fingertip, and then sensed by the photodetector 763.

Meanwhile, a wave (pulse wave) propagating in blood vessels and blood is caused by heartbeats. Thereby, arteries in the human body pulsate, and the thickness of the arteries varies with the pulsation. That is, the pulse wave can be determined as a waveform from the surface of the human body, which reflects a volumetric change caused by the flow of blood into a certain portion of the human body. Accordingly, similar pulsation occurs in arteries in the fingertip inserted in the vital sensor 76, and the blood flow rate varies depending on the pulsation, so that the amount of infrared light absorbed varies with the blood flow rate. Specifically, it is possible to obtain information having similar meaning to heart-rate-related information (e.g., electrocardiographic R-R intervals) by measuring not the movement of heart itself but the movement of blood vessels. Note that electrocardiographic R waves represent contraction of ventricle muscle (i.e., contraction of ventricle), and the number of R waves per minute can be interpreted as the heart rate. An interval between an R wave and a next R wave is an R-R interval. Since the R-R interval is an inverse of the heart rate, the R-R interval being long means that the heart rate is low. Conversely, the R-R interval being short means that the heart rate is high.

Specifically, as the blood flow rate in the inserted fingertip increases, the amount of light absorbed by hemoglobin also increases and therefore the amount of infrared light sensed by the photodetector 763 relatively decreases. Conversely, as the blood flow rate in the inserted fingertip decreases, the amount of light absorbed by hemoglobin also decreases and therefore the amount of infrared light sensed by the photodetector 763 relatively increases.

The light source 762 and the photodetector 763 utilize such an operating principle. The amount of infrared light sensed by the photodetector 763 is converted into a photoelectric signal to detect pulsation of the human body. For example, as shown in FIG. 9, when the blood flow rate in the inserted fingertip increases, the detected value of the photodetector 763 increases, and when the blood flow rate in the inserted fingertip decreases, the detected value of the photodetector 763 decreases. Thus, a portion of a pulse wave in which the value detected by the photodetector 763 increases or decreases is generated as a pulse wave signal. Note that, depending on the circuit configuration of the photodetector 763, a pulse wave signal may be generated such that the detected value of the photodetector 763 decreases when the blood flow rate in the inserted fingertip increases, while the detected value of the photodetector 763 increases when the blood flow rate in the inserted fingertip decreases.

The control section 761 includes, for example, a Micro Controller Unit (MCU). The control sect ion 761 controls the amount of infrared light emitted from the light source 762. The control section 761 also performs A/D conversion on a photoelectric signal (pulse wave signal) outputted from the photodetector 763 to generate pulse wave data (biological information data). Thereafter, the control section 761 outputs the pulse wave data (biological information data) via the connection cable 79 to the core unit 70.

(Summary of Information Processing and Information Processing Program)

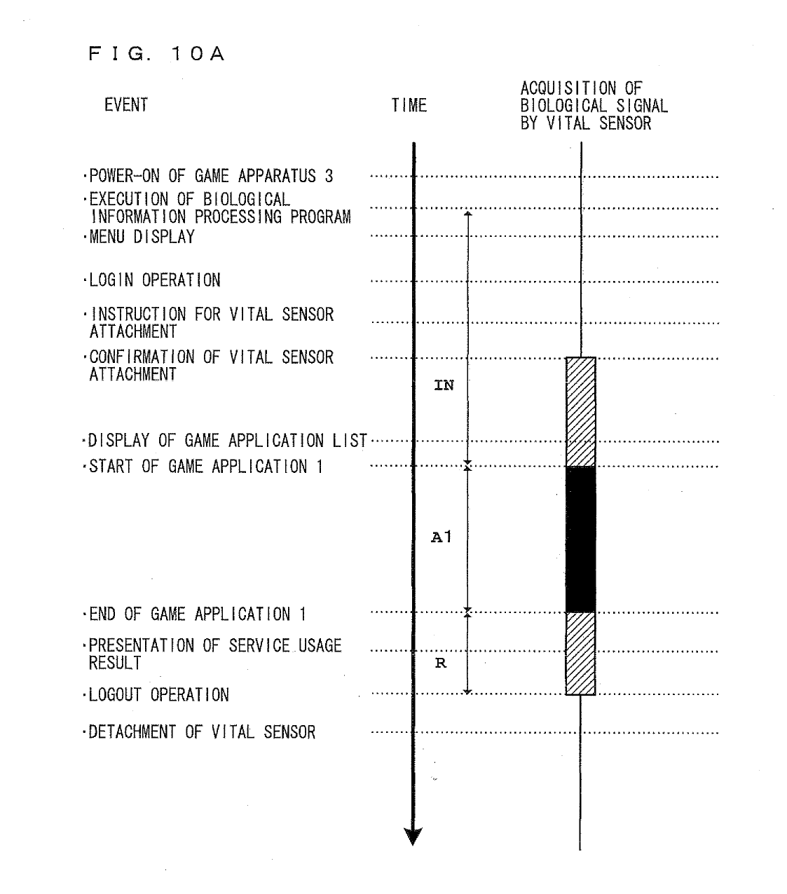

In advance of describing specific processing to be performed by the game apparatus body 5, the following will describe, with reference to FIG. 10A to FIG. 27, a summary of processing to be performed by the game apparatus body 5 and a summary of an information processing program that performs the processing.

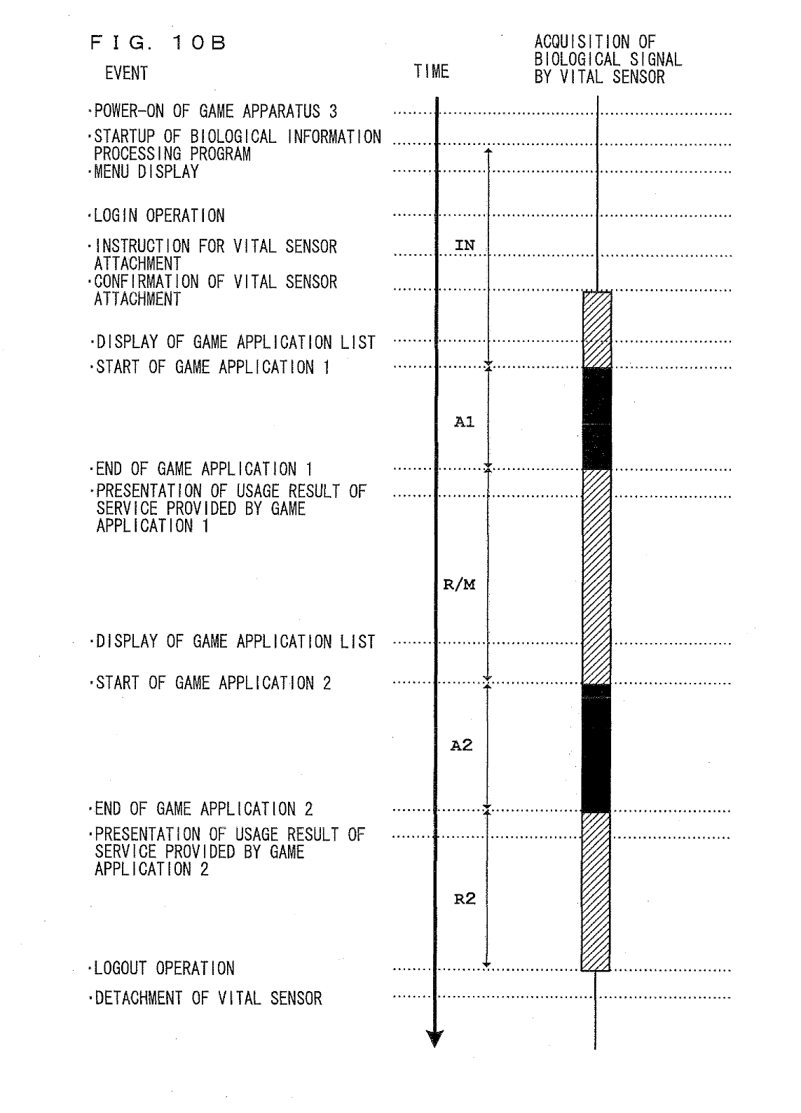

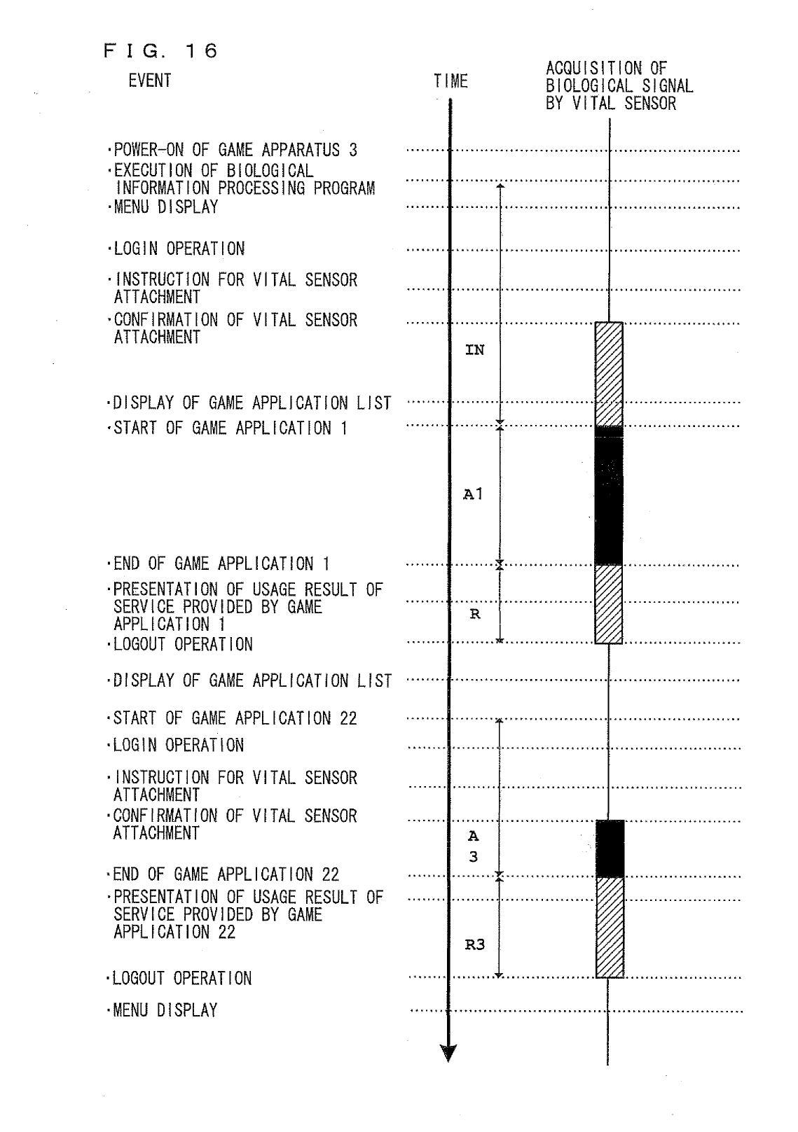

FIGS. 10A, 10B, and 16 are schematic diagrams showing an example of a process to be performed in the present embodiment. FIG. 11 to FIG. 15 and FIG. 17 to FIG. 27 are diagrams showing an example of a series of images to be displayed on the monitor 2.

The information processing program according to an embodiment of the present invention is a biological information processing program which realizes, for each user of the program, a process of acquiring, retaining, and utilizing biological information (including a biological signal, or a biological parameter, or both of them).

This information processing program includes a program (hereinafter, referred to as a program for acquiring a biological signal) for acquiring a biological signal from the user, or a biological parameter derived from the biological signal, or both of them. Moreover, the information processing program includes one or more application programs in addition to the program for acquiring a biological signal.

The one or more applications can be one or more game applications. Typically, such game application can include a process of utilizing, for its game processing, biological signals acquired from its user (s), or biological parameters derived from the signals.

As used herein, the term “game application” means an application for implementing, on a computer system, a sequence of processes in which a user who participates based on specific rules can obtain quantifiable results. The term “application”, or “application program”, or “application software” means a computer program which supports the user to perform a specific kind of work. As used herein, the term “computer system” or “system” means not only a single device but also a plurality of devices, each device being capable of communication with any of other devices.

In the above description, the programs included in the biological information processing program are separated into the “program for acquiring a biological signal” and other one or more (game) application programs. However, each of the (game) application programs may have a function of acquiring a biological signal. The above description means that the biological information processing program has a function of acquiring a biological signal, and each of the (game) application programs may be configured so as to have a function for acquiring a biological signal.

In an embodiment of the present invention, the program for acquiring a biological signal can be executed in a period in which any of the plurality of game applications described above is executed, as well as before and after this period. That is, typically, the information processing program according to the present embodiment is executed so that the period in which the program for acquiring a biological signal is executed includes the period in which the game application utilized by the user is executed.

By allowing the program for acquiring a biological signal to obtain a biological signal from the user even during a period other than the period in which the game application is executed, it is possible to reduce the frequency of newly providing an occasion for acquiring a biological signal or a biological parameter (consequently, causing the user to be aware of the acquisition) during the processing of the game application.

In addition, an acquired biological signal or biological parameter can be utilized, in association with presence/absence of an explicit indication of the acquisition in progress to the user, in order to establish a more tailored way of acquiring biological information from the user. For example, the biological information processing program can compare a biological signal or a biological parameter (e.g., CVRR) acquired. with an explicit indication of the acquisition in progress to the user, with a corresponding biological signal or biological parameter acquired without such indication. The indication to the user can be established via an output device such as the monitor 2. The biological information processing program can also utilize an acquired biological signal or biological parameter for a subsequent process, depending on the presence/absence of such explicit indication. This allows avoiding of a psychological pressure on the user during acquisition of a biological signal or a biological parameter.

Further, when the biological information processing program is allowed to retain a biological signal that has been acquired in a period other than the period in which the game application is executed, a biological parameter adapted for each user can be derived from the biological signal. That is, when a biological signal can be acquired in the above-described manner and the acquired biological signal can be managed in association with the situation where it was acquired, the biological information of the user can be treated multilaterally, thereby realizing provision of services adapted for the user.

Hereinafter, an example of a chronological flow of operation of the biological information processing program will be described with reference to FIG. 10A. FIG. 10A is a schematic diagram showing an example of a chronological process in which an information processing apparatus executing the biological information processing program obtains a biological signal from the user, and retains and utilizes the signal.

Specifically, FIG. 10A is a schematic diagram showing an example of processing of the biological information processing program in the game apparatus body 5. This processing includes a sequence of processes such as acquisition of a biological signal, calculation of a biological parameter based on the acquired biological signal, and presentation, to the user, based on the calculated value. FIG. 10A shows, on its left side, major events along the time axis (a thick line in the center) to be performed after the game apparatus 3 is powered on. Further, FIG. 10A schematically shows, on its right side, the relationship between the events and acquisition of a biological signal by the vital sensor 76.

First of all, when the game apparatus 3 is powered on and the biological information processing program is executed, a list of one or a plurality of services provided by the biological information processing program is displayed on the monitor 2. For example, as shown in FIG. 11, the game apparatus body 5 displays, on the monitor 2, icons (“item 1” and “item 2” in FIG. 11) corresponding to services provided for the user. The user selects a desired service from the list of the services displayed as the icons on the monitor 2. A subsequent process associated with the icon is started on condition that the user performs a selection input operation using the controller 7 or the like.

In the example of FIG. 11, when the icon indicated as the item 1 is selected by the user, a process (user determination process) for causing the user to log into an account which allows the user to refer to a service usage history relating to the user. On the other hand, when the icon indicated as the item 2 is selected by the user, a process for prompting the user to select a game application provided by the biological information processing program is performed instead of the above-described user determination process.

FIG. 10A shows a flow of processing of the biological information processing program, when the icon of “item 1” is selected by the user at the time of menu display described above, and the login process is performed.

When the user has already utilized any of the services provided by the biological information processing program, and a series of usage history data and the like relating to the service are stored in a predetermined storage area so that the program can refer to the data, the usage history data and the like are stored in the storage area as a part of account data associated with an identification code or the like for identifying the user. The user can log into the account by selecting the identification code or the like identifying the user, which is displayed on the monitor 2 (login operation in FIG. 10A). On the other hand, when account data corresponding to the user is not stored in the storage area, the user can form an account which allows the user to utilize his/her usage history and the like.

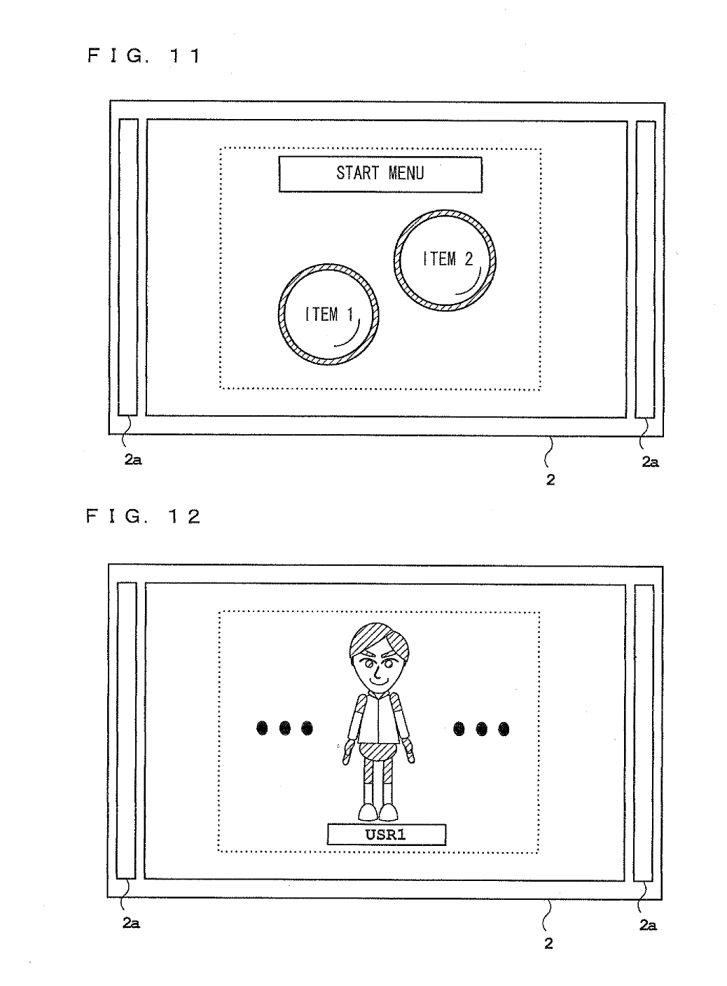

FIG. 12 shows an example of an image displayed on the monitor 2 when the user having the usage history selects the icon of “item 1”, and selects the identification code (USR1 in FIG. 12) for identifying the user. As shown in FIG. 12, not only the identification code but also an expression such as a human-shaped character (avatar) corresponding to the user can be associated with the account.

The usage history data of the user can include all records of all the parameters (including the acquired biological signal and biological parameter) in the biological information processing program. However, part of the previous records might be erased from the storage area included in the game apparatus body 5, at a predetermined timing, depending on the specification of the services provided by the biological information processing program.

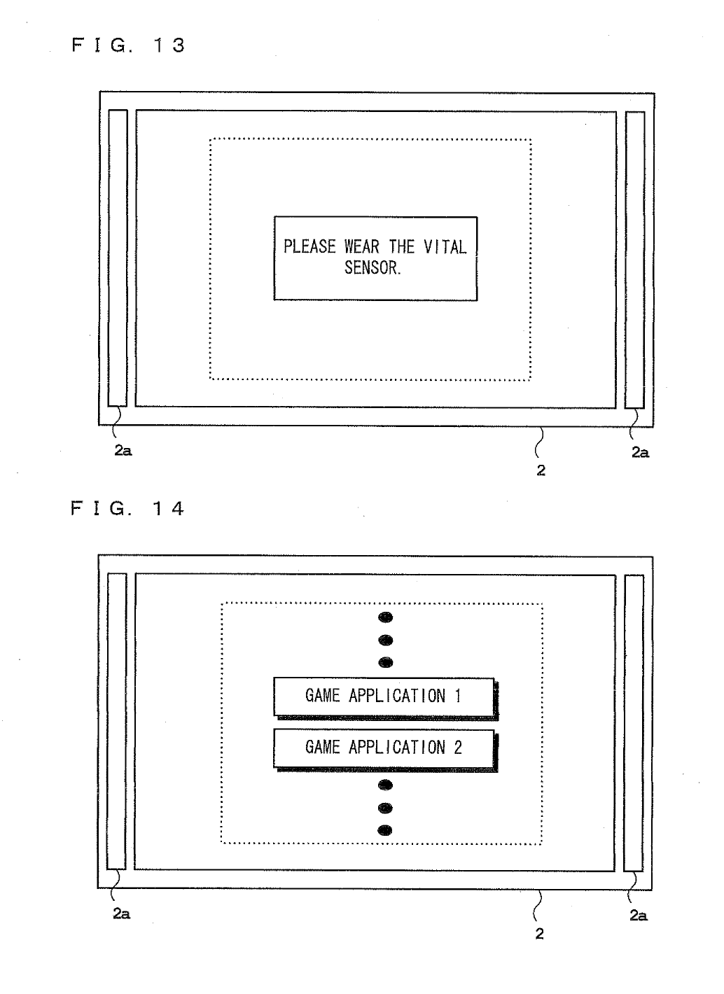

Turning to FIG. 10A, the processing after the user's login operation will be described. After the user's login operation, the game apparatus body 5 prompts, via the monitor 2 or the like, the user to wear the vital sensor 76 which is an input device for acquiring biological information from the user. FIG. 13 shows an example of such prompting for wearing of the vital sensor 76. This prompting may be performed, not only by the display on the monitor 2, but also by other means such as voice which the user can sense, or by a combination thereof.

Next, when an optical system included in the vital sensor 76 confirms that the user has worn the vital sensor 76, the vital sensor 76 obtains a biological signal from the user's body.

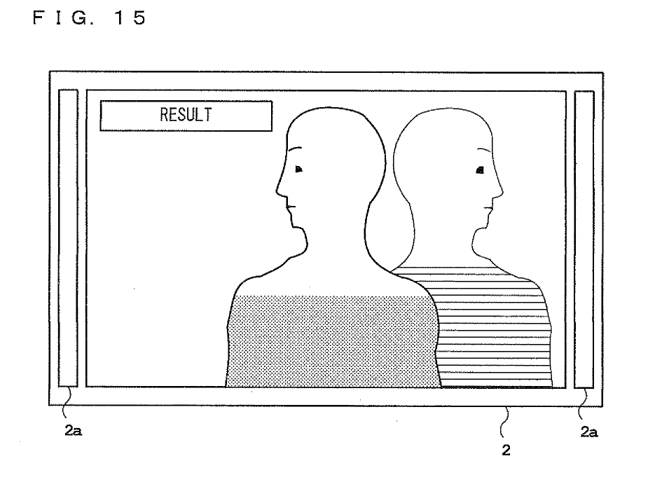

Thereafter, a menu which allows the user to select a service (e.g., game application 1) is presented to the user. For example, as shown in FIG. 14, selectable game applications are displayed as corresponding icons on the monitor 2.

FIG. 10A shows a flow of processing when the icon of “game application 1” is selected by the user at the time of menu display (the example shown in FIG. 14) and thereby the game application 1 is started.

As understood in FIG. 10A, the game apparatus body 5 can obtain a biological signal from the user who wears the vital sensor 76, during a period between the biological information processing program start time and the game application start time. The period is indicated as “IN” period in FIG. 10A. FIG. 10A shows, on the right side, the relationship between the event and the acquisition of a biological signal by the vital sensor 76. In FIG. 10A, a shaded area shows a period during which a biological signal is acquired within the “IN” period.

The game apparatus body 5 can acquire a biological signal from the user via the vital sensor 76, also in a period between the start time of the game application 1 and the end time of the game application 1. The period is indicated as “A1” period in FIG. 10A.

Specifically, in the process of the service provided by the game application 1, the game apparatus body 5 receives an input from the user via the input device including the vital sensor 76. Then, the game apparatus body 5 performs, according to the input, an output (predetermined presentation) to the user via an output device such as the monitor 2. Also in a period (“A1” period) during which the input and the output are alternately repeated, the game apparatus body 5 acquires a biological signal from the user, and derives a biological parameter from the biological signal.

The game application 1 can utilize the biological signal acquired in the “IN” period shown in FIG. 10A, and the biological parameter which is acquired based on the sequence of the biological signals through a predetermined calculation. Although a specific example of the game application and processing thereof will be described later, the game application 1 can utilize the biological signal acquired in the “IN” period and the biological parameter derived from the sequence of biological signals, as values, reference values, threshold values and the like to be used for control of the progression of the game application (in the above example, the progression of the game application during the “A1” period).

In FIG. 10A, rectangle areas on the right side indicate the periods in which the game apparatus body 5 obtains biological signals from the vital sensor 76. Specifically, a shaded rectangle area indicates a period during which a biological signal is acquired within the “IN” period, while a black rectangle area indicates a period during which a biological signal is acquired within the “A1” period.

After the game application 1 is ended, the result obtained through the execution of the game application 1, or the knowledge or the like obtained by referring to the user's usage history, or both of them, are presented to the user (in FIG. 10A, presentation of service usage result). That is, various values obtained through the execution of the game application 1 are outputted to the monitor 2 to be displayed by a presentation manner in which a still image, a moving image, text and the like are combined so that the user can intuitively understand the values. FIG. 15 is a diagram showing an example of a screen display on the monitor 2, in which the result of the game application is displayed in such presentation manner.

In the example shown in FIG. 10A, even after the end of the game application 1, the game apparatus body 5 can obtain a biological signal from the user. In this example, the game apparatus body 5 can obtain a biological signal via the vital sensor 76 from the user who continues to wear the vital sensor 76, even during a period which is after the end of the game application 1 and includes a time when the above-described presentation of the service usage result is performed. This period corresponds to “R” period in FIG. 10A, which is indicated by “shaded area” relating to acquisition of a biological signal by the vital sensor. This acquisition of a biological signal after the end of the game application 1 is ended when the user logs out of the account.

The game apparatus body 5 may be configured as follows. That is, even after the user has performed the logout operation from the account, so long as the user continues to wear the vital sensor 76 and the biological signal acquired from the vital sensor 76 can be associated with the user, the game apparatus body 5 may retain the biological signal acquired after the logout operation, as the user's usage history, so as to refer to and utilize the same for the subsequent information processing.

As described above, the game apparatus body 5 can obtain a biological signal not only in the period (“A1” period) in which the user utilizes the game application 1 but also in the periods before and after the “A1” period (“TN” period and “R” period in FIG. 10A). Further, as shown in FIG. 10B, when the user, after utilization of the game application 1, does not log out of the account and continuously utilizes another application (game application 2), the game apparatus body 5 can obtain a biological signal also in a period (“R/M” period) between the period (“A1” period) in which the user utilizes the game application 1 and the period (“A2” period) in which the user utilizes the game application 2.

In this way, the game apparatus body 5 is given an occasion to acquire a biological signal, even in a period other than the periods in which the game applications (in the above example, the game application 1 and the game application 2) that assume major services to be utilized by the user in the biological information processing program, are executed in the information processing apparatus (in the above example, the game apparatus body 5). Thereby, the game apparatus body 5 can derive a desired biological parameter as necessary. Then, utilizing the acquired various pieces of biological information (including the biological parameter and the biological signal), the game apparatus body 5 progresses the information processing when the game application is executed.

As will be evident from the later description, the biological signals acquired in the above-described respective periods such as the “IN” period, the “A1” period, the “R” period, the “A2” period, and the “R/M” period and the biological parameters derived from the biological signals, are finally (as necessary) stored as biological signal history data Ds21 and biological parameter history data Ds22, in association with phase data Ds22 indicating the periods when these biological signals and parameters were acquired, among account data Ds associated with the user.

Accordingly, the biological signals and the like acquired in the respective periods are stored in the storage area in the game apparatus body 5, in association with the periods when they were acquired. Based on the data thus stored, the game apparatus body 5 progresses the information processing with the various data being differentiated according to the periods when they were acquired.

For example, the game apparatus body 5 may progress the processing of the game application in the “A1” period, based on the biological signal acquired in the “IN” period, or the biological parameter derived from the biological signal, or both of them.

Further, the game apparatus body 5 may progress the processing of the game application in the “A2” period, based on the biological signal acquired in the “IN” period, or the biological parameter derived from the biological signal, or both of them.

Further, the game apparatus body 5 may progress the processing of the game application in the “A2” period, based on the biological signals acquired in the “IN” period and in the “R/M” period, or the biological parameters derived from the biological signals, or both of them.

Depending on the circumstances, the game apparatus body 5 may progress the processing of the game application in the “A2” period, based on the biological signals acquired in the “IN” period, “A1” period, and “R/M” period, or the biological parameters derived from the biological signals, or both of them.

The game apparatus body 5 can select and utilize a biological signal and the like among the biological signals and like acquired in the respective periods, depending on the required type of the information in the processing of the game application. The type is associated with, for example, the situation where the signal was acquired, the acquired biological signal, the acquired biological parameter, or the like.

For example, when a service that tends to activate the sympathetic nervous system is provided in the game application 1 executed in the “A1” period, then if the precision in interpreting the parameter to be taken into consideration during the processing of the game application 2 in the “A2” period can be enhanced by sampling many biological signals acquired when the sympathetic nervous system is activated, the processing of the game application 2 may be performed such that the biological signals and the like acquired in the “A1” period are aggressively utilized.

As described above, according to an embodiment of the present invention, since a biological signal and a biological parameter can be acquired by considering the association with the situations in various scenes, the precision of the subsequent processing to be performed based on the acquired biological signal and biological parameter can be enhanced.

The following will describe an example of the game application to be utilized as the above-described game application 1. This game application can be, for example, a game application which manages a physical exercise aimed at relaxation effect.

While there are various kinds of physical exercises having relaxation effect, the game application 1 provides the user with a game (hereinafter referred to as a stretching game) which introduces, into a progression thereof, user's performing stretching while being aware of his/her breathing and posture.

Biological parameters used for evaluating the relaxation effect may include, but are not limited to, an activity level of parasympathetic nervous system of the user. The activity level of the parasympathetic nervous system of the user is represented based on a coefficient of variance of R-R interval (CVRR) of the user. For example, the CVRR is calculated using cardiac cycles (R-R intervals; refer to FIG. 9) over past 100 pulses indicated by a pulse wave obtained from the vital sensor 76. Specifically, the CVRR is calculated as follows:

CVRR={(the standard deviation of the R-R intervals over 100 pulses)/(the average of the R-R intervals over 100 pulses)}×100

The above-described biological parameter is calculated after the processing of the game application has ended, or in some cases, during execution of the game application, and then it is presented to the user. The biological parameter is presented to the user in a presentation manner which the user can easily understand. For example, the CVRR described above is represented as “relax fluid” which is a virtual object fluid, and a variation in the CVRR is represented by a variation in a surface level of the relax fluid poured into in a container. An image in which the states before and after the variation (e.g., before and after the execution of the game application 1) are compared, or a moving image showing the manner of the variation can be presented to the user.

Hereinafter, a summary of the processing of the stretching game will be described, starting from selection of the game application (as for an example of contents displayed on the monitor 2, refer to FIG. 14) in the process flow shown in FIG. 10A.

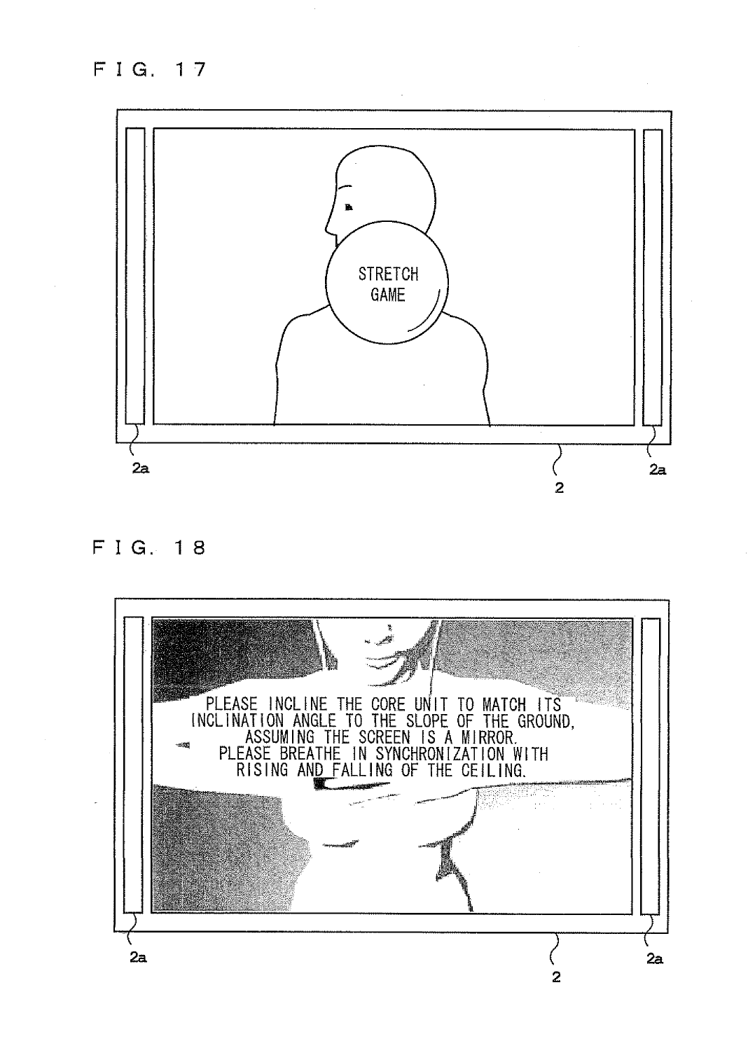

When the user selects the game application 1 from the list of game applications shown in FIG. 14, the game apparatus body 5 displays, on the screen of the monitor 2, that the stretching game (game application 1) is started. FIG. 17 shows an example of the screen display on the monitor 2 at this time.

When the stretching game is started, the monitor 2 displays an explanation about an operation posture and an operation method which the user should know when playing the game. FIG. 18 shows an example of contents displayed on the monitor 2.

In the example of FIG. 18, a model wears the vital sensor 76, and takes a posture to hold the core unit 70 with both hands with both elbows sticking out leftward and rightward in a longitudinal direction of the core unit 70. The monitor 2 displays this posture as an operation posture for the stretching game. Then, the monitor 2 displays an operation method, i.e., “Please incline the core unit 70 to match its inclination angle to the slope of the ground, assuming that the screen is a mirror.” The monitor 2 also displays an operation method, i.e., “Please breathe in synchronization with rising and falling of the ceiling.” Thus, the user can know the operation posture and the operation method for the stretching game, such as those shown in FIG. 13, by viewing those displayed on the screen.

As shown in FIG. 19, in the stretching game, for example, a game is performed in which a player character PC moves based on a biological signal (a pulse wave signal) of the user and the motion and posture (inclination of the core unit 70) of the user. The player character PC is required to fly in a space (e.g., a cave) between a ceiling T and a ground B which are, for example, scrolled from the left to the right in the virtual game world. In this case, the ceiling T and the ground B are obstacles in the way of the player character PC flying in the space. The player character PC includes a first player character PC1, and a second player character PC2 provided on the first player character PC1, which are separable.

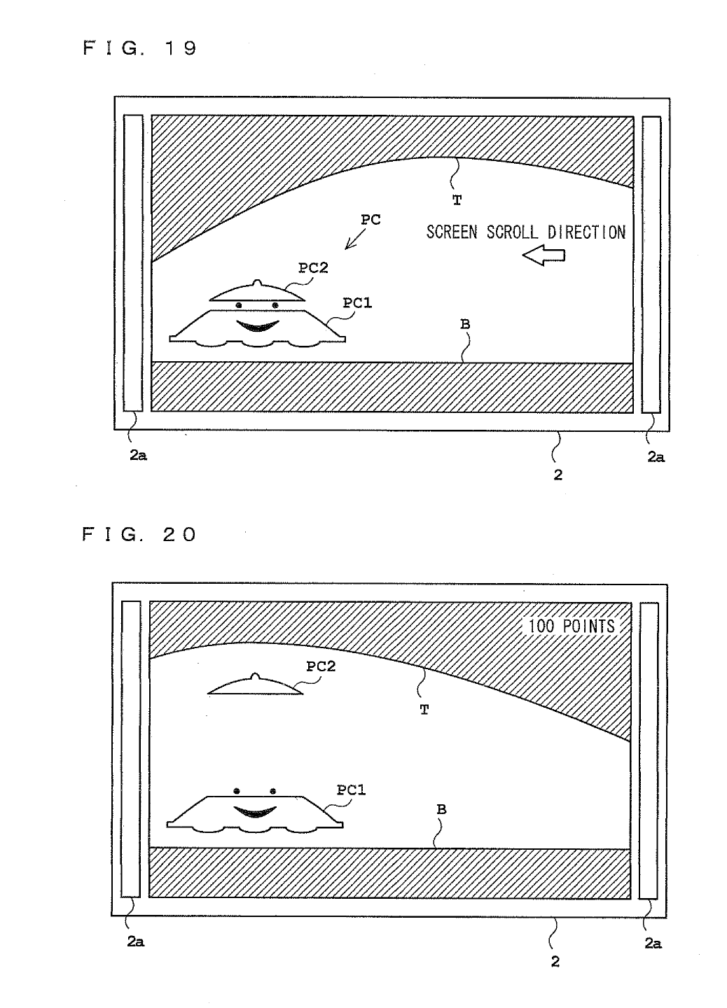

In FIG. 20, the second player character PC2 can be moved up, where a maximum height of the second player character PC2 is limited to the height of the ceiling T with respect to the first player character PC1. Here, the second player character PC2 is moved up and down, depending on the respiratory state of the user. For example, the second player character PC2 is moved up with respect to the first player character PC1 when the user exhales, and is moved down toward the first player character PC1 when the user inhales. In this embodiment, the heart rate HR of the user is calculated using the pulse wave signal, and if the heart rate HR is increasing, it is determined that the user is inhaling, and if the heart rate HR is decreasing, it is determined that the user is exhaling. The heart rate HR is defined as the number of times the heart beats to pump blood in a minute (60 seconds) (bpm; beats per minute). In this embodiment, the heart rate HR is calculated by dividing 60 (seconds) by a cardiac cycle (R-R interval; e.g., time (seconds) from a minimum value of a pulse wave to a next minimum value, or time (seconds) from a rising edge of a pulse wave to a next rising edge; refer to FIG. 9).

Rising and falling of the ceiling T are controlled based on the respiration frequency of the user. At this time, the game apparatus body 5 can utilize the biological signal acquired from the user during the “IN” period before the start of the stretching game (game application 1) as well as the biological parameter (e.g., an average of respiration frequencies in the “IN” period) derived from the sequence of the biological signals, as values, reference values, and the like to be used for control of the progress of the game application 1. Thereby, the game application body 5 can obtain the biological information of the user under the state different from the state where the user performs the game application 1, and thus the game application body 5 can obtain the information for multilaterally taking the characteristics of the user.

Specifically, in this embodiment, the game apparatus body 5 calculates an average of respiration frequencies in the “IN” period (hereinafter referred to as a reference respiration frequency), based on the biological signal (the pulse wave signal) acquired from the vital sensor 76 in the “IN” period. It is generally known that the heart rate is increased with inspiration (i.e., the R-R interval is shortened) while it is decreased with expiration (i.e., the R-R interval is lengthened). Of course, the respiration frequency strongly characterizes the fluctuation of the heartbeat (or pulse). The game apparatus body 5 obtains the respiration frequency from the rising/falling frequency of the heart rate HR of the user, and calculates an arithmetic average of the respiration frequencies obtained in the “IN” period to obtain a reference respiration frequency. Then, the respiration frequency within a predetermined range, based on the obtained reference respiration frequency, is utilized as a range of the parameter for controlling the provided virtual game world, in order to provide the user with relaxation effect.

Initially, the game apparatus body 5 indicates, to the user, a respiration frequency (or a respiration rate corresponding to the respiration frequency) which the user should take at the start of the game application 1 (stretching game). At this time, a respiration frequency indicated to the user is set to be, for example, higher than the reference respiration frequency. That is, at the start of the stretching game, the user is required by the game apparatus body 5 to breathe faster than his/her breathing in the “IN” period (e.g., to breathe at a rate of 120% relative the breathing during the “IN” period). This predetermined respiration frequency is presented to the user as the height of the ceiling T in the virtual game world displayed on the monitor 2.

On the other hand, the game apparatus body 5 also sets a respiration frequency which the user should take at the end of the stretching game. At this time, a respiration frequency indicated to the user is set to be, for example, lower than the reference respiration frequency. That is, at the end of the stretching game, the user is required by the game apparatus body 5 to breathe slower than his/her breathing during the “IN” period (e.g., to breathe at a rate of 80% relative to the breathing during the “IN” period). Further, a rate of change from the value of the respiration frequency at the start to the value of the respiration frequency at the end is also determined.

Next, the game apparatus body 5 calculates the current respiration frequency of the user, based on the frequency at which the heart rate HR of the user rises and falls, and changes the height of the ceiling T in the virtual game world at the change rate that is prescribed based on the value of the reference respiration frequency, while monitoring the current respiration frequency. The stretching game of this example adopts a scoring system in which, if the second player character PC2 contacts the ceiling T in the virtual game world, the score of the stretching game is decreased. Accordingly, when the game apparatus body 5 varies the height of the ceiling T with the progression of the game and presents the same to the user, this presentation influences the manner of breathing of the user.

Here, the game apparatus body 5 adjusts the rising/falling frequency of the ceiling T so that the respiration frequency presented to the user is gradually decreased with the progression of the game (i.e., so that the breathing of the user is gradually slowed) and finally reaches a respiration frequency lower than the average value of the respiration frequencies of the user obtained in the “IN” period. That is, by moving up and down the second player character PC2 with the rising and failing of the ceiling T that is adjusted by the game apparatus body 5, the user is required to breathe at a rate of 80% relative to his/her breathing during the “IN” period. That is, the user is required to gradually slow down the breathing. As a result, with the progression of the stretching game, the user breathes, at the start of the stretching game, at a rate higher than his/her usual breathing, and then breathes at the usual rate, and finally breathes at a rate lower than the usual breathing.

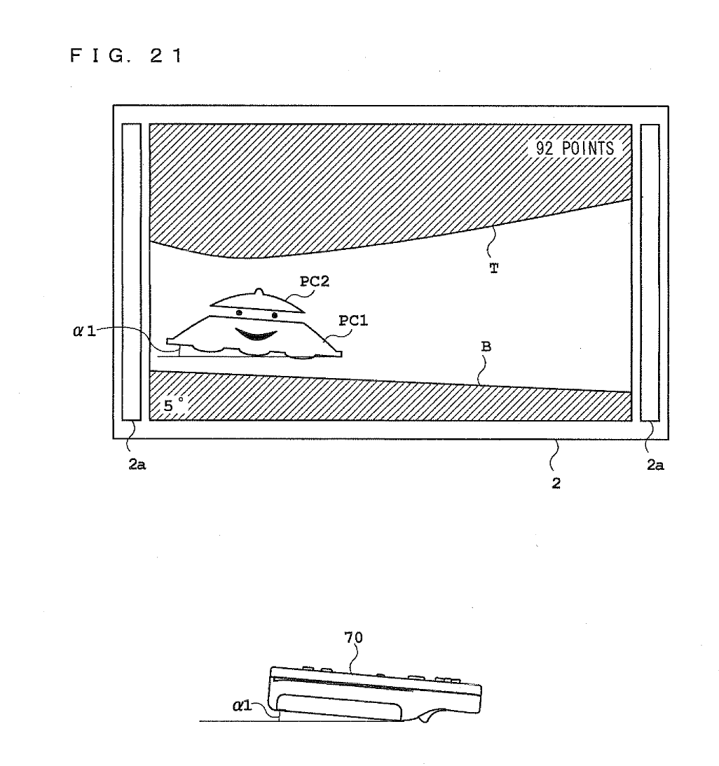

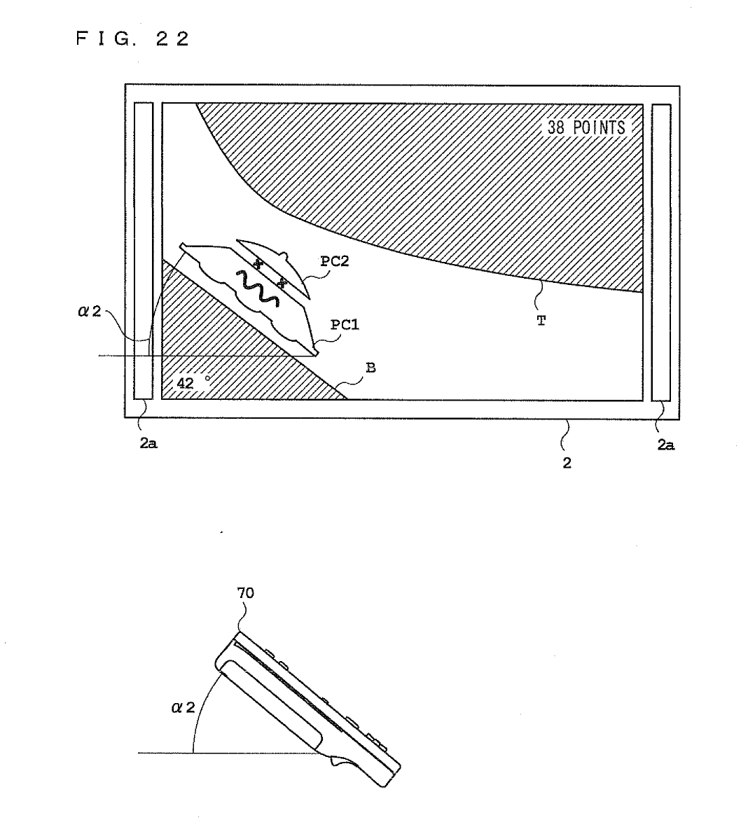

With reference to FIG. 21, the player character PC can fly obliquely along the ground B. Here, the player character PC has its flying attitude, depending on the inclination of the core unit 70. For example, when the user in the operation posture as shown in FIG. 18 inclines the core unit 70 to the right at an angle of α1 toward the monitor 2, the displayed player character PC is also inclined to the right at an angle of α1 in synchronization with the act of inclining the core unit 70. Also, as shown in FIG. 22, when the user in the operation posture as shown in FIG. 18 inclines the core unit 70 to the right at an angle of α2 toward the monitor 2, the displayed player character PC is also inclined to the right at an angle of α2 in synchronization with the act of inclining the core unit 70. In other words, the user feels like he/she inclines the player character PC by inclining the core unit 70.

The stretching game of this example adopts a scoring system in which, if the second player character PC contacts the ground B in the virtual game world, the score of the stretching game is decreased. Accordingly, when the game apparatus body 5 varies the inclination of the ground B with the progression of the game, this variation acts on the posture of the user.

FIG. 22 shows the state where the inclination angle of the ground B is increased with the progression of the stretching game.

When the inclination angle of the ground B is increased, the user has to incline the core unit 70 at an angle similar to the slope of the ground B so as to incline the player character PC to match the inclination angle to the slope of the ground B. In other words, the user is required to do stretching movement, such as bending or twisting a portion of his/her body at which the core unit 70 is held or attached.

When the user feels that the requested stretching movement is difficult to do, the slope of the ground B is fixed to an inclination angle at this time. For example, in the present embodiment, a pulse wave amplitude PA (e.g., a difference in height between a maximum value of a pulse wave and a next minimum value; refer to FIG. 9) obtained from the pulse wave signal is used as a parameter to evaluate how the user feels of the posture and movement taken at the current time. Then, the game apparatus body 5 varies, according to the result of evaluation, the condition (e.g., face color, countenance, or the like) of the player character PC displayed on the monitor 2.

In the example of FIG. 21, the ground B inclined to the right at an inclination angle of 5° is displayed. When the user, facing the monitor 2, inclines the core unit 70 to the right at an angle of α1 (e.g., 5°) to match the inclination angle to the slope of the ground B, the displayed player character PC is also inclined to the right at an angle of α1 (e.g., 5°) in synchronization with the user's act of inclining the core unit 70. In this case, since it is still easy for the user to take such posture, the displayed player character PC has calm countenance.

On the other hand, in the example of FIG. 22, the ground B inclined to the right at an inclination angle of 42° is displayed. When the user, facing the monitor 2, inclines the core unit 70 to the right at an angle of α2 (e.g., 42°) to match the inclination angle to the slope of the ground B, the displayed player character PC is also inclined to the right at an angle of α2 (e.g., 42°) in synchronization with the user's act of inclining the core unit 70. In this case, the user has much difficulty in holding such posture, so that the displayed player character PC has painful countenance.

Such a difference in representation between the player character shown in FIG. 21 and the player character shown in FIG. 22 is realized by the game apparatus body 5 using the following references. For example, when the pulse wave amplitude PA of the user is secured at 90% or more as compared to that at the start of the stretching game, the game apparatus body 5 determines that “the user has no difficulty.” Further, when the pulse wave amplitude PA of the user is reduced to 50% to 90% as compared to that at the start of the stretching game, the game apparatus body 5 determines that “the user has difficulty.” Moreover, when the pulse wave amplitude PA of the user is reduced to 50% or less as compared to that at the start of the stretching game, the game apparatus body 5 determines that “the user has much difficulty.” When the pulse wave amplitude PA of the user reaches 50% or less, the game apparatus body 5 determines that the condition of the user at this time is a limit for the user, and regards the inclination angle at this time (limit inclination angle) as a measure for evaluating the pliancy of the user's body.

When the game application 1 (stretching game) is ended, the game apparatus body 5 can present, to the user, the values of the biological parameter obtained before and after the stretching game, in an appropriate form, via the monitor 2 or the like. Thereby, the user can intuitively recognize the effect of the stretching game provided by the biological information processing program.

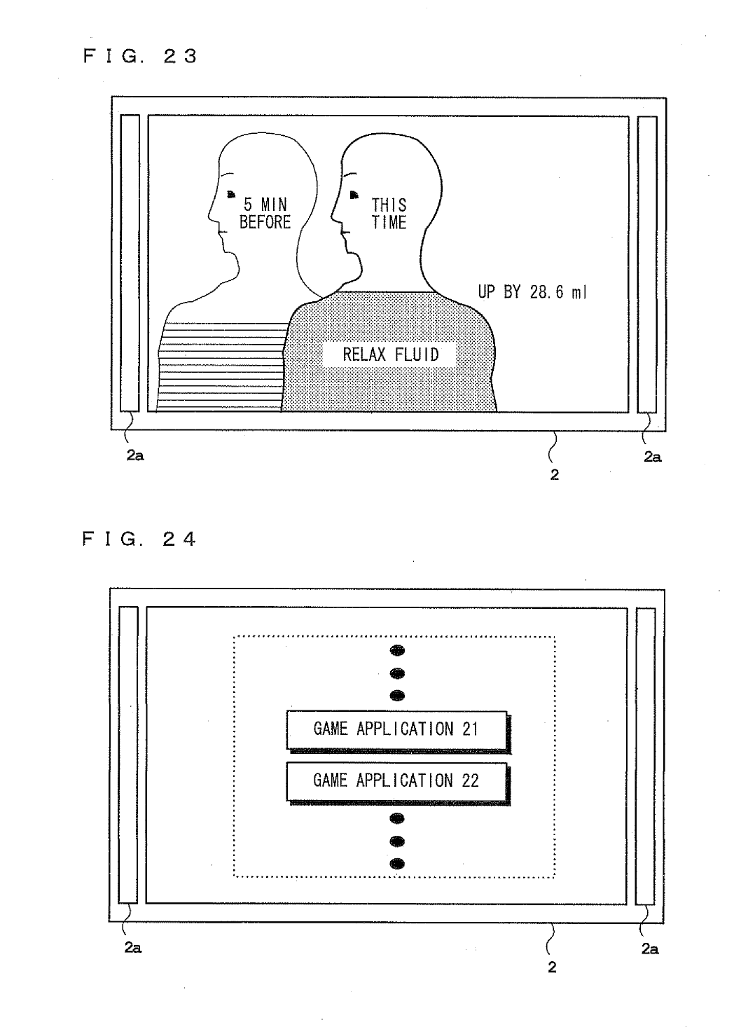

FIG. 23 is a diagram showing a display on the monitor 2 in which the amount of “relax fluid” before the stretching game (denoted as “5 min before” in FIG. 23) is displayed as well as the amount of “relax fluid” after the stretching game, in order to cause the user to recognize his/her parasympathetic nerve activity before and after the stretching game. Further, an increase in the relax fluid amount after the stretching game is displayed as a numerical value. For example, in this embodiment, a CVRR of the user before the stretching game is compared with that after the stretching game, and a value obtained by multiplying a difference therebetween by 10 is displayed as an increase/decrease in volume (ml).

Further, a value indicating the degree of the pliancy of the user's body may be displayed after the end of the stretching game. For example, the above-described limit inclination angle is used to display the degree of the user's pliancy (pliancy score). Specifically, the user's limit inclination angle is compared with an ideal inclination angle in the stretching game, and the degree of the user's pliancy (pliancy score) is calculated and displayed based on a difference therebetween.

Up to here, the description has been made of an example of execution of the biological information processing program through the process in which the icon indicated as the item 1 is selected by the user, and the user logs into the account which allows the user to refer to the service usage history relating to the user, in the example shown in FIG. 11.

On the other hand, when the icon of “item 2” shown in FIG. 11 is selected and a process of prompting the user to select the service provided by the biological information processing program is performed without going through the above-described login process, the game apparatus body 5 displays, as shown in FIG. 24, icons corresponding to game applications (in FIG. 24, game application 21 and game application 22) to be selected by the user, on the monitor 2.

The user selects a desired item (e.g., game application 22) from the selectable game applications displayed on the monitor 2, by using an input device such as the controller 7, and thereby processing relating to the selected game application is started.

Here, the user selects the game application (in this example, the game application 22) in a condition where the user logs out of his/her account. After the processing of the game application 22 is started, the user can log into an account for reading account data Ds which allows the user to refer to the usage history. This account data Ds is an account common to that managed when the user selects the item 1 and performs login in the example of FIG. 11.

Accordingly, the user's usage history including the biological information (including the biological signals acquired during the “IN” period, “A1” period, “R” period and the like in FIG. 10A or FIG. 10B, and the biological parameters derived from the biological signals) which is acquired in the process of utilizing the game application (e.g., the game application 1) which is provided as the result of the user's selection of the item 1 in FIG. 11, can be referred to also in the game application provided via the account information even when the user selects the item 2.

In the above-described configuration, for example, when the game application 22 which is selected to be utilized by the user who is identified by an identification code “USR1”, is a game application that utilizes the biological information acquired from the user, this game application 22 can provide the user with a service more adapted to the user, by referring to, in its game processing, the biological information which has been stored in the process in which the user USR1 utilizes the game application 1.

For example, it is possible to use, as the game application 22, a game application in which the characteristics, in the settings of the game application, assigned to a player character to be operated by the user are determined in association with a biological signal acquired by the biological information processing program and/or a biological parameter based on the biological signal.

The following will describe, with reference to FIG. 16, a process in which the user selects the item 1 to utilize the game application 1, logs out of the game application 1, and then utilizes the game application 22. FIG. 16 is a schematic diagram showing the chronological process of executing the biological information processing program.

The first half of FIG. 16 is identical to the sequence of events shown in FIG. 10A. That is, the flow from the “IN” period to the “R” period in FIG. 16 is identical to that shown in FIG. 10A. That is, FIG. 16 shows processes to be performed after the user's selecting the icon of “item 1” on the menu display shown in FIG. 11, the login process, the process relating to the game application 1, the process such as display of the result, and the logout process.

Specifically, FIG. 16 shows a process flow in which the process in the game apparatus body 5 returns to the menu display (FIG. 11) after the “R” period has been ended (i.e., after the logout operation), and then the user selects the “item 2”. When the “item 2” is selected by the user, the game apparatus body 5 displays a menu screen as shown in FIG. 24 on the monitor 2. When the user selects the game application 22, game processing thereof is started (the “A3” period in FIG. 16 is started). During the game processing, the game apparatus body 5 prompts the user to log into the account, and the login allows the game apparatus body 5 to refer to the usage history. By referring to the usage history, the game apparatus body 5 can provide the user with services adapted to the user. Meanwhile, during the “A3” period, the game apparatus body 5 prompts the user to wear the vital sensor 76, and thereafter, obtains a biological signal from the user.

The game application 22 is, for example, a so-called shooting game as described below. A summary of the shooting game will be described with reference to FIG. 25 to FIG. 27. Of course, such shooting game can be used as the game application 1 in the biological information processing program.

With reference to FIG. 25, the monitor 2 displays a virtual game world in which a player character PC3 and enemy characters EC are arranged. The player character PC3 is moved in the virtual game world in accordance with an operation performed on the operation section 72 (e.g., the cross key 72a) in the core unit 70. Further, the player character PC3 discharges “discharge object” (e.g., a bullet B) in the virtual game world in accordance with the biological information based on the pulse wave data obtained from the vital sensor 76.

Specifically, when the game apparatus body 5 detects a pulse timing of the user (e.g., a timing of heart contraction; a timing exactly at which blood vessels in a portion of the user's body to which the vital sensor 76 is attached contract or expand), the player character PC3 discharges a predetermined number of bullets B. When a bullet B hits any of the enemy characters EC, an endurance of the enemy character EC is decreased depending on an attack power of the bullet B. When the attack power of the bullet B having hit the enemy character EC exceeds the endurance of the enemy character BC, the enemy character EC disappears from the virtual game world.

In the following description, a bullet B is used as an example of a discharge object with which the player character PC3 attacks the enemy characters EC. Here, the “discharge object” is used as a term expressing an object which is discharged from the player character PC3 to attack the enemy characters EC. The discharge object may include a bullet, a shell, a bomb, a grenade, a rocket, a missile, a ball, an arrow, a beam, a laser beam, and the like in the virtual game world.

The configuration of the discharge object which is discharged, at every pulse timing, from the player character 203 may be changed based on the interval of pulse timings (the interval of heartbeats) of the user.

As a first example, the number of bullets B to be discharged at every pulse timing from the player character PC3, and the discharge direction, are changed based on the interval of pulse timings (the interval of heartbeats) of the user. For example, when the heart rate HR of the user is lower than a predetermined first threshold value, the player character PC3 discharges a bullet B1 per pulse timing of the user (refer to FIG. 25).

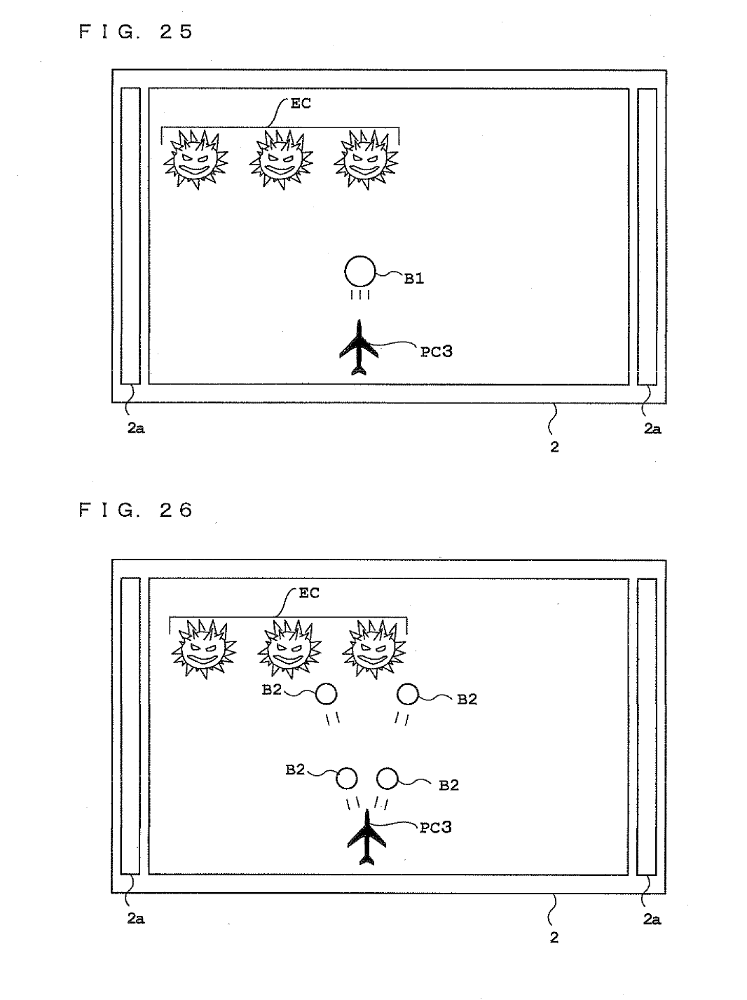

At this time, the player character PC3 discharges a bullet B1 in a forward direction from the front of the player character PC3 (an upward direction in FIG. 25; hereinafter referred to as discharge direction A). When the heart rate HR of the user is equal to or higher than the first threshold value and is lower than a predetermined second threshold value, the player character PC3 discharges two bullets B2 per pulse timing of the user (refer to FIG. 26). At this time, the player character PC3 discharges two bullets B2 forward each at a predetermined angle relative to the directly forward direction of the player character PC3 (hereinafter referred to as discharge direction B).

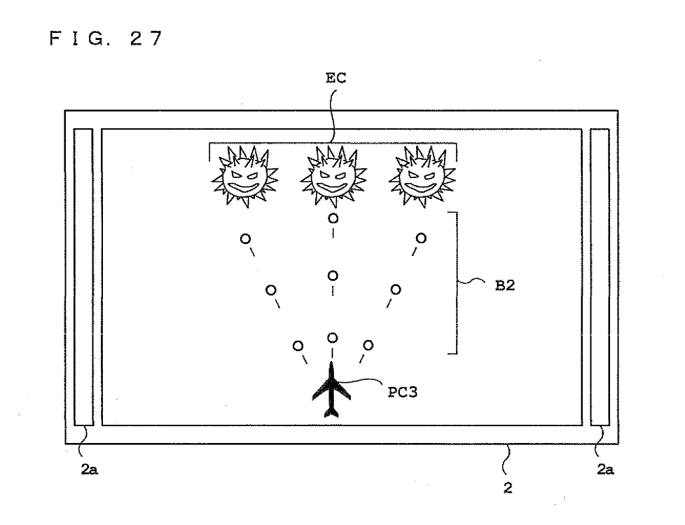

When the heart rate HR of the user is equal to or higher than the second threshold value and is lower than a predetermined third threshold value, the player character PC3 discharges three bullets B3 per pulse timing of the user (refer to FIG. 27). At this time, the player character PC3 discharges three bullets B3 forward, one to the forward direction of the player character PC and the remaining two each at a predetermined angle relative to the directly forward direction of the player character PC3 (hereinafter referred to as discharge direction C). When the heart rate HR of the user is equal to or higher than the third threshold value, the player character PC3 discharges five bullets B5 per pulse timing of the user. At this time, the player character PC3 discharges five bullets B5 forward in an angle range wider than that for three bullets B3, where the front direction from the player character PC is set as the center of the angle range (hereinafter referred to as discharge direction D).

As a second example, the attack power, to the enemy character EC, of the bullet B discharged from the player character PC3 is changed depending on the interval of pulse timings (the interval of heartbeats) of the user. For example, when the heart pulse HR of the user is lower than a predetermined first threshold value, the player character PC3 discharges a bullet B1 having a highest first attack power (refer to FIG. 25). When the heart pulse HR of the user is equal to or higher than the first threshold value and is lower than a predetermined second threshold value, the player character PC3 discharges a bullet B2 having a second attack power lower than the first attack power (refer to FIG. 26). When the heart pulse HR of the user is equal to or higher than the second threshold value and is lower than a predetermined third threshold value, the player character PC3 discharges a bullet B3 having a third attack power lower than the second attack power (refer to FIG. 27). When the heart pulse HR of the user is higher than the third threshold value, the player character PC3 discharges a bullet B5 having a fourth attack power lower than the third attack power.

As described above, the player character PC3 discharges the discharge object in accordance with the pulse timing of the user (e.g., a timing of heart contraction; a timing exactly at which blood vessels in a portion of the user's body to which the vital sensor 76 is attached contract or expand), resulting in a highly entertaining shooting operation which the user cannot easily anticipate. On the other hand, when the configuration of the discharge object to be discharged per pulse timing of the user (the number of the discharge objects, the attack power of the discharge object, the discharge direction of the discharge object, or the like) is changed based on the heart rate HR of the user, i.e., the interval of pulse timings (the interval of heartbeats) of the user, a further highly entertaining shooting operation which the user cannot easily anticipate can be realized.

In the above-described configuration, the mariner of determining the first threshold value, the second threshold value, or the third threshold value can be made dependent on the biological parameter derived from the biological signal acquired during a period (e.g., “IN” period) other than the period during which the shooting game is executed. A procedure of a specific process thereof will be described later. By determining a threshold value to be used for a subsequent game application which utilizes the biological information of the user, based on pieces of biological information which have been acquired during various preceding process periods in the biological information processing program, it is possible to provide a service of the subsequent application with settings more appropriate for the user of the application.

(Details of Information Processing)

The following will describe, in detail, information processing (particularly, biological information processing) to be performed in the game system 1.

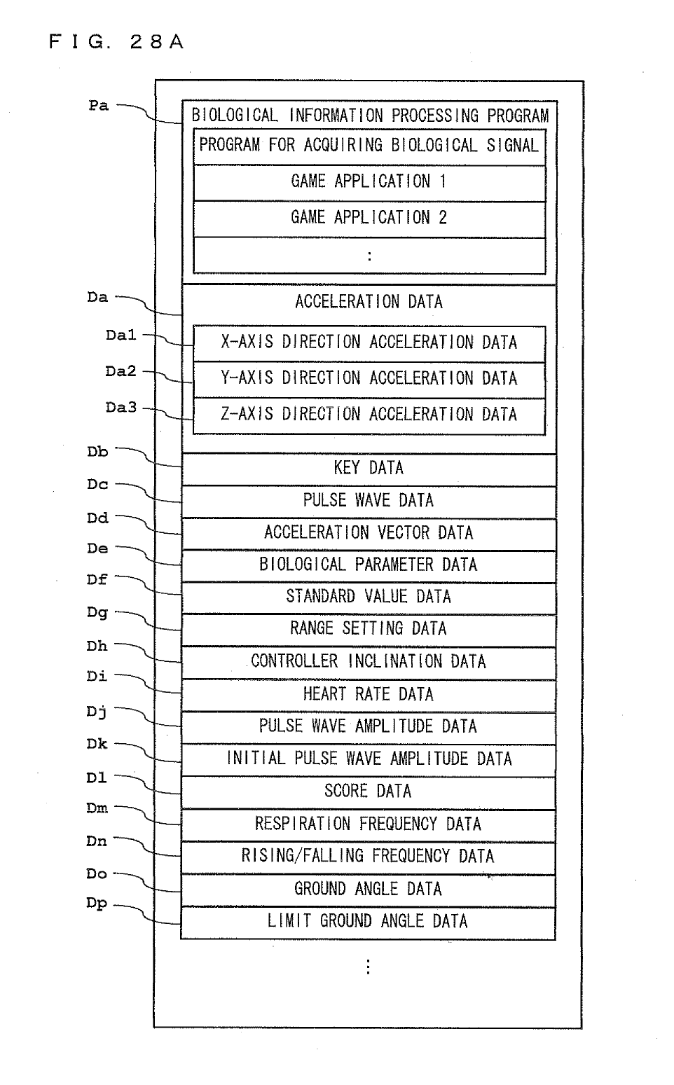

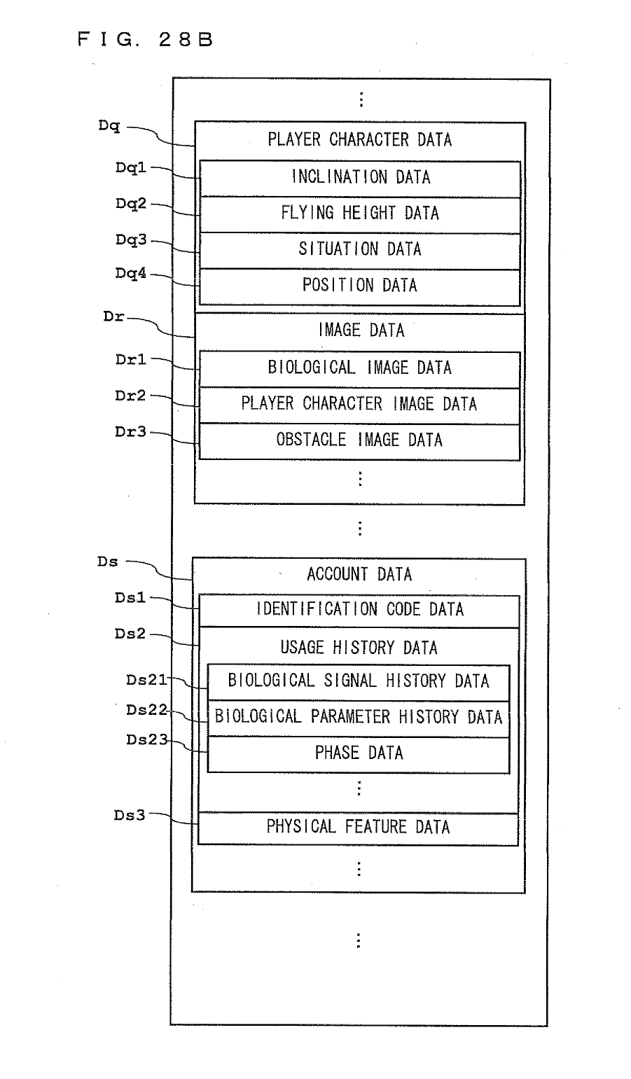

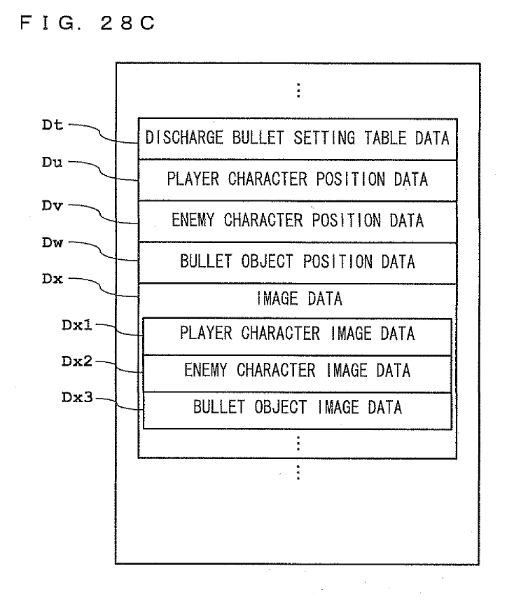

First of all, with reference to FIG. 28, main data used in the information processing will be described. FIG. 28 is a diagram showing an example of main data and programs stored in the external main memory 12 and/or the internal main memory 35 (hereinafter the two main memories is collectively referred to as a main memory) of the game apparatus body 5.

(Main Data Used in Biological Information Processing)

As shown in FIGS. 28A to 26C, a data storage area of the main memory stores data as follows.

That is, the data storage area of the main memory stores acceleration data Da, key data Db, pulse wave data Dc, acceleration vector data Dd, biological parameter data De, standard value data Df, range setting data Dg, controller inclination data Dh, heart rate data Di, pulse wave amplitude data Dj, initial pulse wave amplitude data Dk, score data Dl, respiration frequency data Dm, rising/falling frequency data Dn, ground angle data Do, limit ground angle data Dp, player character data Dq, image data Dr, account data Ds, and the like. The data storage area of the main memory further stores discharge bullet setting table data Dt, player character position data Du, enemy character position data Dv, bullet object position data Dw, image data Dx, and the like.

Note that the main memory stores, in addition to the data included in the information shown in FIG. 28, data required for game processing, such as data (position data, etc.) relating to objects and the like which appear in the game and other than the player character PC, data (background data, etc.) relating to the virtual game world, and the like.

Moreover, the program storage area of the main memory stores various programs Pa constituting the biological information processing program.

The acceleration data Da indicates an acceleration of the core unit 70. Acceleration data included in a series of pieces of operation information which are transmitted as transmission data from the core unit 70 is stored as the acceleration data Da in the main memory. The acceleration data Da includes X-axis direction acceleration data Da1 indicating an acceleration which is detected with respect to an X-axis component by the acceleration sensor 701, Y-axis direction acceleration data Da2 indicating an acceleration which is detected with respect to a Y-axis component, and Z-axis direction acceleration data Da3 indicating an acceleration which is detected with respect to a Z-axis component. Note that the wireless controller module 19 included in the game apparatus body 5 receives acceleration data included in operation information transmitted in predetermined cycles (e.g., 1/200 sec) from the core unit 70, and stores the acceleration data into a buffer (not shown) included in the wireless controller module 19. Thereafter, the acceleration data stored in the buffer is read out on a frame-by-frame basis (one frame corresponds to a game processing cycle (e.g., 1/60 sec)), and the acceleration data Da in the main memory is updated with the acceleration data.

In this case, the cycle of reception of the operation information is different from the processing cycle, and therefore, a plurality of pieces of operation information received at a plurality of timings are stored in the buffer. In the following description of the process, it is assumed that only the latest one of a plurality of pieces of operation information received at a plurality of timings is invariably used to perform processing in each step described below, before control proceeds to the next step.

Although, in a process flow described below, the acceleration data Da is updated on a frame-by-frame basis (one frame corresponds to the game processing cycle), the acceleration data Da may be updated in other process cycles. For example, the acceleration data Da may be updated in transmission cycles of the core unit 70, and the acceleration data Da thus updated may be used in game processing cycles. In this case, the cycle in which the acceleration data Da1 to Da3 are stored as the acceleration data Da is different from the game processing cycle.

The key data Db indicates that the operation sections 72 of the core unit 70 each have been operated. Key data included in a series of pieces of operation information which are transmitted as transmission data from the core unit 70 is stored as the key data Da into the main memory. Note that a method of updating the key data Db is similar to that of the acceleration data Da and will not be described in detail.

The pulse wave data Dc indicates a pulse wave signal having a required time length, which is obtained from the vital sensor 76. Pulse wave data included in a series of pieces of operation information which are transmitted as transmission data from the core unit 70 is stored as the pulse wave data Dc into the main memory. Note that a history of a pulse wave signal having a time length required in a process described below is stored as the pulse wave data Dc into the main memory, and is appropriately updated in response to reception of operation information.

The acceleration vector data Dd indicates an acceleration vector which is calculated using an acceleration indicated by the X-axis direction acceleration data Da1, the Y-axis direction acceleration data Da2, and the Z-axis direction acceleration data Da3. Data indicating a direction and a magnitude of an acceleration applied to the core unit 70 is stored as the acceleration vector data Dd into the main memory.

The biological parameter data De indicates various biological parameters of the user at the present time, and amounts derived from the values of the parameters. For example, data indicating an amount of “relax fluid” which is calculated based on a CVRR of the user in a period from the present time back to a predetermined point in the past is also stored as the biological parameter data De.

The standard value data Df indicates a standard value of biological information (e.g., parasympathetic nerve activity under a specific situation) for each age, which is previously statistically calculated.

Control parameters required for controlling execution of game applications of the biological information processing program are stored in the range setting data Dg. The range setting data Dg is a storage area to be used for defining a value which is required to vary within a predetermined range with reference to a value derived from a biological signal or the like. For example, the value is a parameter whose maximum value and minimum value are defined by the derived value, and which attenuates at a constant rate (change rate) between the maximum value and the minimum value.

The controller inclination data Dh indicates an inclination of the core unit 70 with respect to a direction of gravity. The heart rate data Di indicates a history of heart rates HR of the user (e.g., a value obtained by dividing 60 sec by a cardiac cycle (R-R interval)) over a predetermined period of time. The pulse wave amplitude data Dj indicates a history of pulse wave amplitudes PA of the user over a predetermined period of time. The initial pulse wave amplitude data Dk indicates a pulse wave amplitude PA of the user which is calculated at the start of the stretching game.

The score data Dl indicates a score in the stretching game. The respiration frequency data Dm indicates a respiration frequency of the user. The rising/falling frequency data Dn indicates a rising/falling frequency of the ceiling T in the stretching game, which is calculated depending on the respiration frequency of the user. The ground angle data Do indicates an inclination angle of the ground B in the stretching game. The limit ground angle data Dp indicates a limit inclination angle of the ground B for the user in the stretching game.

The player character data Dq relates to the player character PC, and includes inclination data Dq1, flying height data Dq2, situation data Dq3, and position data Dq4. The inclination data Dq1 indicates an inclination angle of the player character PC which is inclined depending on an inclination of the core unit 70. The flying height data Dq2 indicates a height to which the second player character PC2 is moved up with respect to the first player character PC1. The situation data Dq3 indicates a face color or countenance of the player character PC corresponding to a difficulty or easiness level of the user. The position data Dq4 indicates a position of the player character PC in the virtual game world.

The image data Dr includes biological image data Dr1, player character image data Dr2, obstacle image data Dr3, and the like. The biological image data Dr1 is used to display biological information of the user on the monitor 2. The player character image data Dr2 is used to generate a game image of the virtual game world in which the player character PC is arranged. The obstacle image data Dr3 is used to generate a game image of the virtual game world in which obstacles (the ceiling T and the ground B) are arranged.

The account data Ds includes usage history data and the like which are associated with a code (identification code) for identifying each user who utilizes the biological information processing program.

Specifically, the account data Ds includes identification code data Ds1 for identifying a user, and usage history data Ds2, physical feature data Ds3 and the like which are associated with the identification code data Ds1. The usage history data Ds2 includes biological signal history data Ds21, biological parameter history data Ds22, and phase data Ds23.

The biological signal history data DS21 is a series of biological signal data which have been acquired and stored in association with the user who utilizes the data. The biological signal history data Ds21 includes, not only data obtained during execution of the game application, but also biological signals obtained during periods, other than the execution of the game application, in which biological signals can be acquired from the vital sensor 76. The biological parameter history data Ds22 includes a series of biological parameter data derived from the biological signals which have been acquired. The phase data Ds23 is data for associating the biological signal history data Ds21 and the biological parameter history data Ds22 with the situations where these data were obtained. More specifically, the phase data Ds23 includes, as necessary, data for discriminating the periods (e.g., “TN” period, “A1” period, “R/M” period, and the like) in which the biological signal history data Ds21 and the like were obtained. Further, the physical feature data Ds3 indicates physical features of the user (e.g., age, gender, height, weight, and the like) which have been inputted at the time of account formation.

When the various data obtained during execution of the biological information processing program are stored as the above-described usage history data Ds2, all the obtained various data may be stored in the storage area of the game apparatus body 5 as the usage history data Ds2. Alternatively, only data, among the obtained various data, which meet predetermined conditions may be stored as the usage history data Ds2 (for example, among the biological signals sampled from the vital sensor 76, an initial value at the start of the sampling, and values sampled at every time point elapsed by a predetermined period from the start of the sampling, may be stored).

Further, discharge bullet setting table data Dt, player character position data Du, enemy character position data Dv, bullet object position data Dw, and image data Dx, which are described below, are data to be used in a shooting game to be described later.

The discharge bullet setting table data Dt is predetermined table data (refer to Table 1 described later) used for setting the number of bullets B to be discharged from the player character 203, the attack power of the bullets B, and the discharge direction.

The player character position data Du indicates the position of the player character PC3 in the virtual game world.

The enemy character position data Dv indicates the position of each of the enemy characters EC in the virtual game world. The bullet object position data Dw indicates the position of each of the bullets B in the virtual game world.

The image data Dx includes player character image data Dx1, enemy character image data Dx2, bullet object image Dx3, and the like.

(Flow of Specific Information Processing)

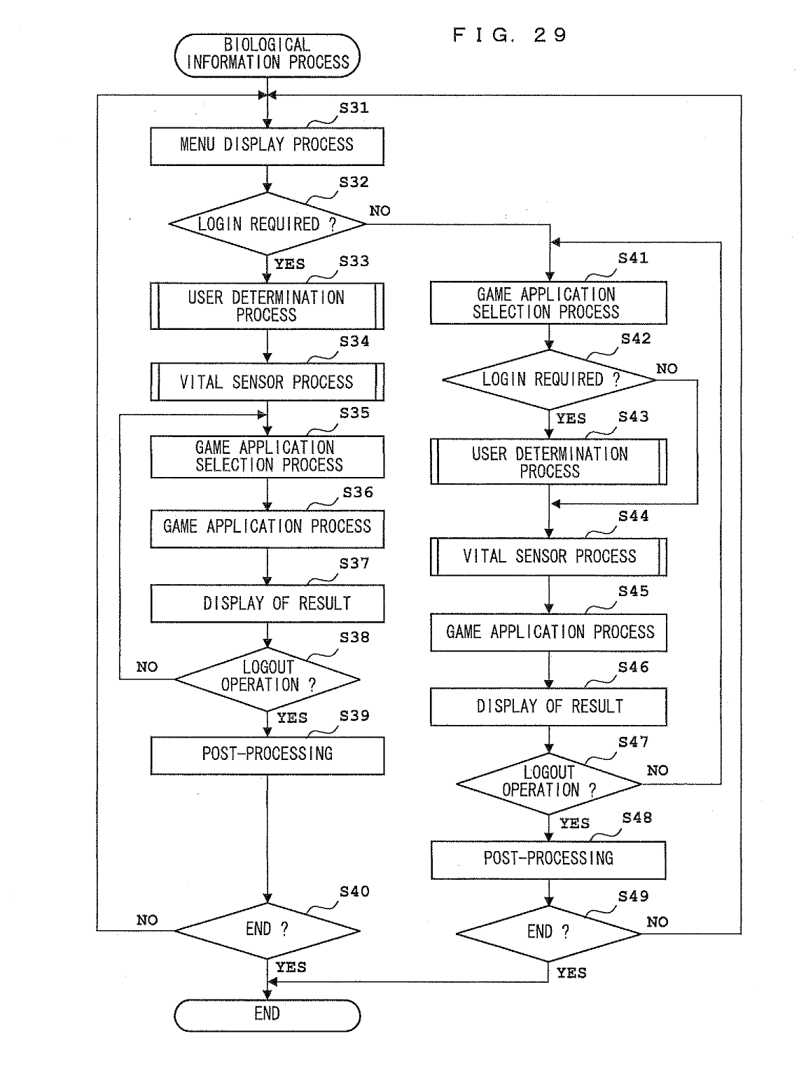

Next, information processing to be performed in the game apparatus body 5 will be described in detail with reference to FIGS. 29 to 33.

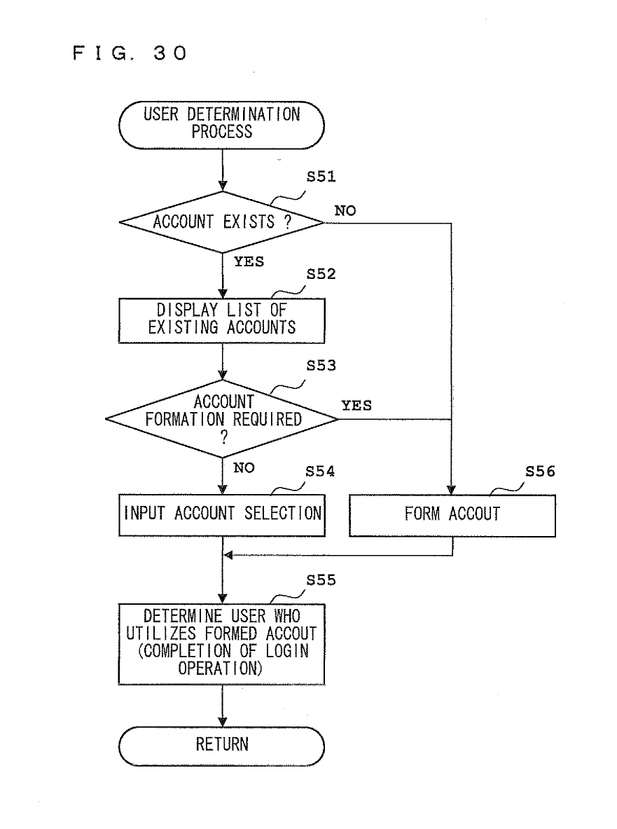

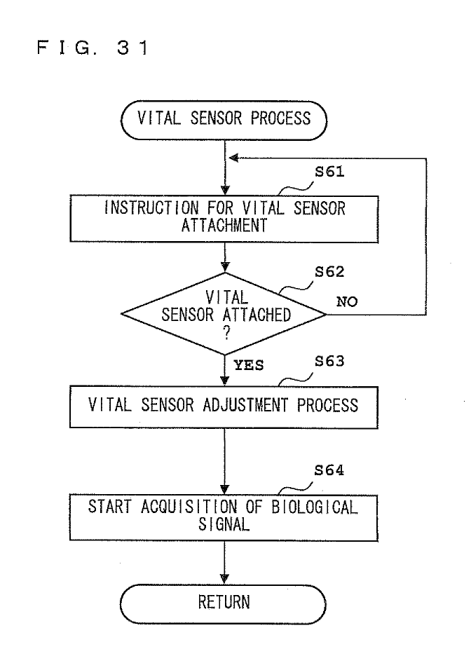

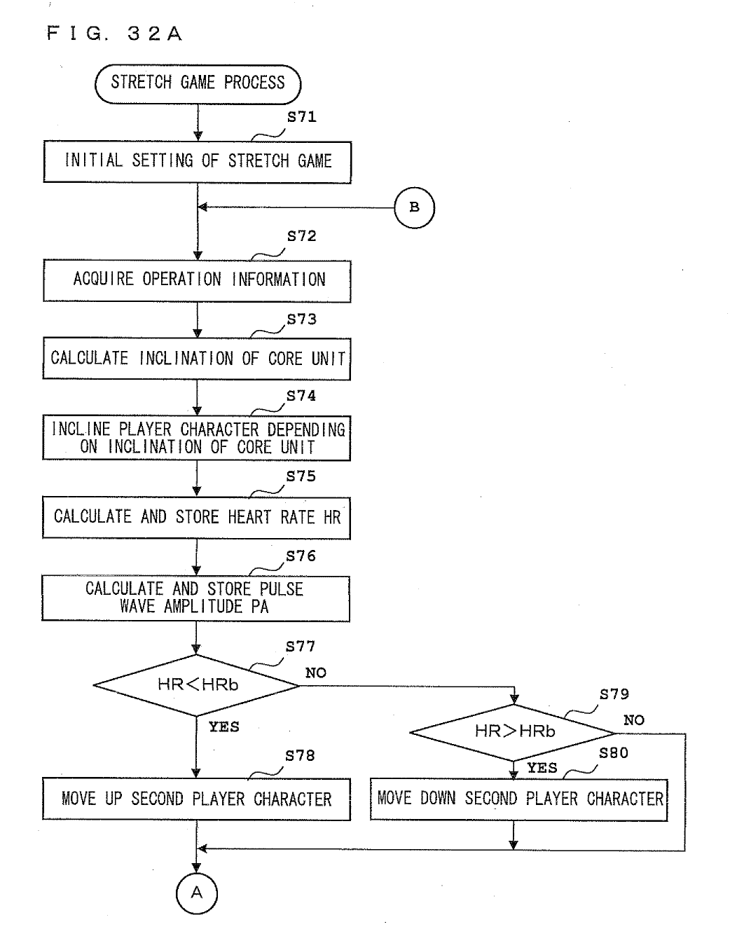

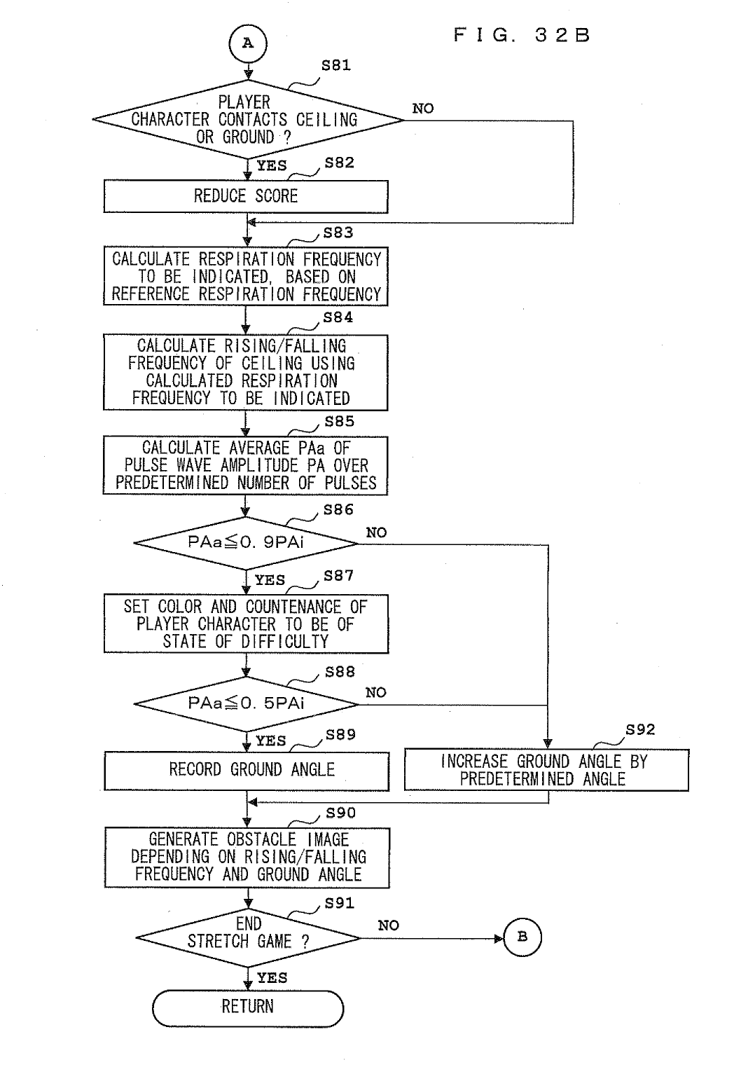

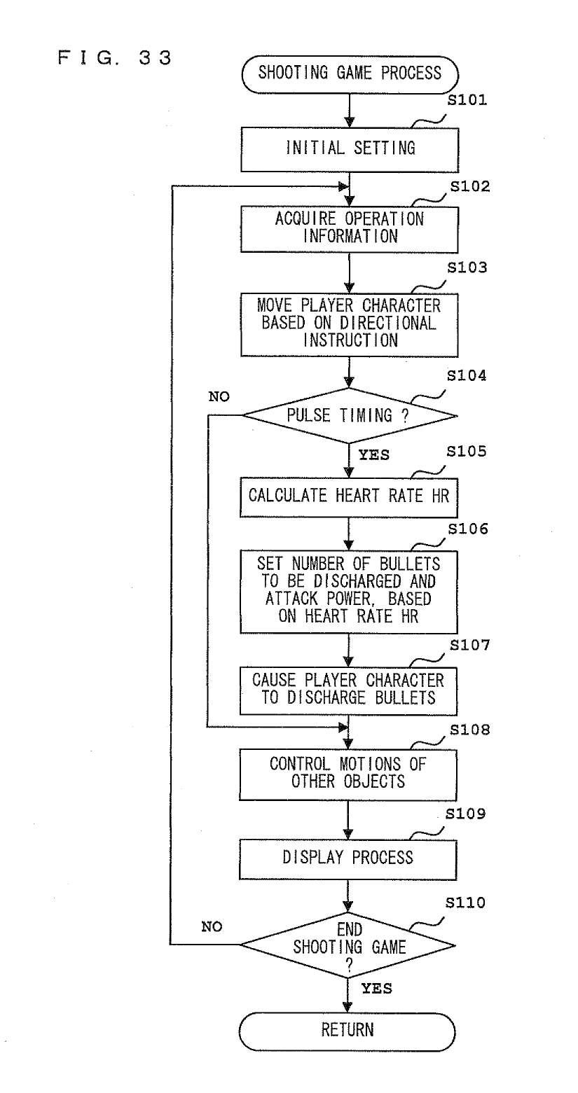

FIG. 29 is a flowchart showing an example of information processing to be performed in the game apparatus body 5. FIG. 30 is a flowchart showing a flow of a subroutine “user determination process” shown in the flowchart of FIG. 29. Likewise, FIG. 31 is a flowchart showing a flow of a subroutine “vital sensor process” shown in the flowchart of FIG. 29. FIGS. 32A and 32B are flowcharts showing an example of a process of “stretching game” that is provided as an available game application in the process of executing the biological information processing program. Further, FIG. 33 is a flowchart showing an example of a process of “shooting game” that is provided as such a game application. Note that, in the figures showing the flowcharts among the figures attached hereto, “step” is abbreviated to “S”.

The processes shown in the flowchart of FIG. 29 are performed after the game apparatus body 5 is powered on and a sequence of initial processes are performed. That is, when the game apparatus body 5 is powered on, the CPU 10 of the game apparatus body 5 executes a boot program stored in the ROM/RTC 13, thereby initializing the respective units such as the main memory and the like. Thereafter, the biological information processing program stored in the optical disc 4 is read into the main memory, and execution of the biological information processing program is started by the CPU 10.

The processes shown in the flowchart of FIG. 29 are achieved by executing a plurality of programs constituting the biological information processing program, in cooperation with each other.

First of all, in step 31, the CPU 10 displays, on the monitor 2, a list of one or a plurality of services which the biological information processing program can provide to the user. For example, as shown in FIG. 11, the CPU 10 displays icons corresponding to the provided services (in FIG. 11, “item 1” and “item 2”). The user selects a desired one from the list of the services indicated by the icons, by using an input device such as the controller 7.

Next, in step 32, the CPU determines whether or not the process associated with the item selected by the user is a process to be started from the user's logging into the account for referring to the usage history or the like of each user.

In the example of FIG. 11, the icon indicated as the item is associated with that the user logs into the account that allows the user to refer to the usage history of the service relating to the user. That is, when the user selects the item 1, the CPU 10 determines, in step 32, that the user needs to log into the account (YES in step 32), and goes to step 33.

On the other hand, the icon indicated as the item 2 is associated with that the user selects a desired one from among game applications which the biological information processing program can provide (game application selection process in step 41). That is, when the user selects the item 2, the CPU 10 determines, in step 32, that the user need not log into the account at this stage (NO in step 32), and goes to step 41.

In step 33, the CPU 10 performs a user determination process. Hereinafter, the user determination process will be described with reference to FIG. 30. FIG. 30 is a flowchart showing a flow of the user determination process.

In step 51 shown in FIG. 30, the CPU 10 determines, with reference to the account data Ds, whether or not there has been any user who has formed an account in the process of utilizing the biological information processing program.

When a user who has formed an account exists (YES in step 51), the CPU 10 goes to step 52. In step 52, the CPU 10 displays a list of existing accounts. This list of existing accounts may be displayed, as shown in FIG. 12, in such a manner that the account is associated with a human-shaped character (avatar) corresponding to the user, as well as with an identification code (USR1 in FIG. 12) of the user.

When the user can find his/her account in the list of existing accounts, the account which allows the user to refer to his/her usage history already exists in the list, and therefore, the user need not form a new account (corresponding to “NO” in step 53). At this time, the CPU 10 determines the user who utilizes the service provided by the biological information processing program (step 55: completion of login operation) on condition that the user selects an icon or the like indicating his/her account from the list by using an input device such as the controller 7 and performs an appropriate input (step 54).

On the other hand, when the user does not find his/her account in the list of existing accounts (corresponding to “YES” in step 53), the CPU 10 provides the user with a process for forming a new account (step 56). After formation of a new account is completed, the CPU 10 stores the formed account as an account corresponding to the user, automatically or upon an additional input by the user. Thus, the user who utilizes the service provided by the biological information processing program is determined (step 55: completion of login operation).

In the process of forming the new account, the CPU 10 can request, as necessary, the user to input information unique to the user. For example, pieces of information such as user's height, weight, age, gender, and the like are input by the user, and stored as physical feature data Ds3. The physical feature data Ds3 can be referred to if needed during the process of the biological information processing program.

When the user determination process is completed, the CPU 10 executes a next process, i.e., a vital sensor process (step 34 in FIG. 29).

Hereinafter, the vital sensor process (step 34 in FIG. 29) will be described with reference to FIG. 31. In this process, the CPU 10 prompts the user to wear the vital sensor 76, and starts to obtain a biological signal from the user via the vital sensor 76 when various conditions for acquiring a biological signal from the user are satisfied. FIG. 31 shows a flowchart (steps) illustrating an example of the vital sensor process.

First of all, in step 61, the CPU 10 prompts, via the monitor 2, the user to attach the vital sensor 76 to a predetermined portion of his/her body. As already described above, FIG. 13 shows an example of an instruction which prompts the user to wear the vital sensor 76.

Next, in step 62, the CPU 10 determines, via an optical system provided in the vital sensor 76, whether or not the user has attached the vital sensor 76 as prompted. Attachment of the vital sensor 76 is performed when, for example, the user inserts a portion of the body (e.g., a fingertip) in a space between the light source 762 and the photodetector 763 in the vital sensor. More specifically, when the user inserts a fingertip in a space between the light source 762 and the photodetector 763 in the vital sensor, the optical system including the light source 762 and the photodetector 763 senses the presence of the inserted fingertip, based on that a level of light received by the photodetector 763 reaches a predetermined value. A signal indicating this sensing is transferred to the CPU 10 via the control section 761 in the vital sensor 76. Thereby, the CPU 10 determines that the user has attached the vital sensor 76 to the portion of the body.

When attachment of the vital sensor 76 is confirmed (YES in step 62), the CPU 10 goes to the next step (step 63). When attachment of the vital sensor 76 is not confirmed (NO in step 62), the CPU 10 continues to instruct the user to attach the vital sensor 76.

In step 63, in order to obtain appropriate conditions for acquiring a biological signal from the attached vital sensor 76, the CPU 10 performs various adjustment processes (e.g., adjustment of light amount in the light source 762) in cooperation with the control section 761 in the vital sensor 76. Thereafter, in step 64, the CPU 10 starts a sequence of processes for acquiring a biological signal. The principle of acquisition of a biological signal by the vital sensor 76 and the operation based on the principle have already been described.

Turning to FIG. 29, the following will describe the processes subsequent to the vital sensor process (corresponding to step 34 in FIG. 29) which has been described with reference to FIG. 31.

First of all, in step 35, the CPU 10 presents, to the user, game applications which are available services for the user, via the monitor 2 or the like. Next, the CPU 10 determines a game application to be executed, in accordance with a selection of the user by an input operation using an input device such as the controller 7. As already described, FIG. 14 shows an example of the display on the monitor 2 in which the selectable game applications are displayed as corresponding icons.

Next, in step 36, execution of the game application selected by the user is started. The selected game application defines specific rules relating to a sequence of processes in which the user can participate via an input device such as the controller 7. Then, the CPU 10 executes the sequence of processes in which the user participates according to the rules to obtain a quantifiable result. Although the already-described stretching game is an example of the selectable game application, the present invention is not restricted thereto. The internal processes of the stretching game will be described in detail later.

Further, in step 37, the CPU 10 presents, to the user, the result obtained in the game application execution process shown by step 36, via the monitor or the like. A specific manner of presentation will be described later in the description of the game application process exemplifying the stretching game.

Thereafter, in step 38, the CPU 10 asks the user to select whether or not to log out of the account. In this step 38, when the user selects to continue the state where the user logs into the account (NO in step 38), the CPU 10 again executes the above-described game application selection process (step 35) and the subsequent processes. Thereby, the user is allowed to reutilize other game applications including the first selected game application. On the other hand, when, in step 38, the user selects not to continue the state where the user logs into the account (YES in step 38), the CPU 10 goes to the next step (step 39) to perform a logout process (from the account) or the like.

In step 39, the CPU 10 stores, as necessary, the various history data to which the user is logging in, into the account data Ds (refer to FIG. 28B) corresponding to the user, and proceeds the process to cause the user to log out of the account. At this time, also stored in the account data Ds are the usage history relating to the game application utilized by the user (including the biological signals and the biological parameters acquired during execution of the game application), and the biological signal and the biological parameter acquired during a period which is between the login operation and the logout operation and is other than the period in which the game application is executed. The history data relating to the biological signals and the biological parameters, and the information about the periods in which these data were acquired, are stored as the biological signal data Ds21, the biological parameter data Ds22, and the phase data Ds 23, respectively, in association with the identification code data Ds1 of the user.

Even after the user has performed the logout operation from the account, so long as the user continues to wear the vital sensor 76 and the biological signal acquired from the vital sensor 76 can be associated with the user, the game apparatus body 5 may retain the biological signal acquired after the logout operation so that it can refer to the biological signal in the user's usage history to utilize the same for the subsequent information processing.

Next, in step 40, the CPU 10 performs, on the monitor 2 or the like, a display which prompts the user to select whether or not to end utilization of the biological information processing program. The user selects, using the controller 7, one of options displayed on the monitor 2. When the user selects to end utilization of the biological information processing program (YES in step 40), the program ends all the processes thereof. When the user selects to continue utilization of the biological information processing program (NO in step 40), the program again goes to the menu display process in step 31.

As descried above, in step 31, the CPU 10 displays, on the monitor 2, a list of one or a plurality of services which the biological information processing program can provide to the user.

Up to here, the description has been made of the process according to FIG. 11 in the case where the user selects the icon of “item 1” which is associated with the user's login operation to the account.

The following will describe a process flow in a case where, when the user selects the icon of “item 2” in the example shown in FIG. 11, the CPU 10 proceeds the game application selection process (step 35) without going through the process relating to the user's login operation (NO in step 32).

Even when the CPU 10 returns to the process in step 31 without ending execution of the biological information processing program in step 40, the user is allowed to go to the processes associated with the icons of “item 1” or “item 2”, from the “menu display process” in step 31.

Although the game application selection process in step 41 may be varied depending on the contents of services provided to the user as game applications, the fundamental process flow thereof is identical to the game application selection process described in step 36. For example, when the process of prompting the user to select one of the services provided by the biological information processing program is performed in step 41, the CPU 10 displays, on the monitor 2, the icons corresponding to the game applications to be selected (in this example, game application 21 and game application 22) as shown in FIG. 24. Then, the user asks for starting utilization of a desired game application by selecting an icon corresponding to the game application on the monitor 2 by using an input device such as the controller 7.

In step 42, the CPU 10 determines whether or not the service of the game application selected by the user is a service which requires, when starting the process thereof, the user to log into the account for referring to the usage history or the like of each user. This process defined in step 42 is fundamentally identical to the process defined in step 32.

However, in the process flow from step 41 to step 47, when the user does not perform a logout operation in step 47 and returns to the game application process defined in step 41, then if the user utilizing the game application continues to utilize the game application, the user determination process (step 42) can be skipped (NO in step 42) because the user needs not to again log into the account in step 41.

The user determination process (step 43) and the vital sensor process (step 44) which follow step 42 are identical to the user determination process (step 33) and the vital sensor process (step 34) which follow step 32, respectively.

Accordingly, the details of the processes in steps 42 to 44 will be skipped here, but the following point will be additionally described.

In the process flow shown in FIG. 29, not only the usage history data relating to the game application utilized by the user (including the biological signal and the biological parameter obtained during execution of the game application) but also a biological signal and a biological parameter acquired during a period which is between the login operation and the logout operation and is other than the period in which the game application is executed, can be utilized in the process flow shown in FIG. 29, on condition that the user determination process (i.e., the user's login operation) defined in step 33 or step 43 is executed. The history data associated with the user are stored in the account data Ds (refer to FIG. 28B).

Accordingly, for example, when the CPU 10 returns to the process in step 31 without ending execution of the biological information processing program in step 40, then if the game application process in step 45 utilizes the biological signal and/or the biological parameter, the CPU 10 can progress the game application process by utilizing the biological signal and/or the biological parameter acquired in the processes from step 33 to step 38.

(Example of Game Application Using Biological Information: Stretching Game)

The following will describe more specific processing of a game application in a case where the user selects “stretching game” as game application 1 in step 35 in the process of the biological information processing program shown in FIG. 29.

In this case, there has been established a state where, in the vital sensor process in step 34, a biological signal is acquired from the user via the vital sensor 76, and further, the biological signal data is appropriately transferred from the vital sensor 76 to the game apparatus body 5.

Accordingly, the biological signal has already been acquired from the vital sensor 76 before the “stretching game” is selected by the user as a game application to be utilized (step 35 in FIG. 29) and is executed in the game application process (step 36 in FIG. 29). Since the stretching game is performed based on the predetermined biological information acquired from the previously acquired biological signal, the user enjoys, from the game application, the service that is customized with the biological information unique to the user being reflected to the service.

Hereinafter, the process flow of the stretching game to be executed under the above-described conditions will be described with reference to FIG. 32A and FIG. 32B.

In step 71, the CPU 10 initializes settings for the stretching game process, and goes to the next step.

In the stretching game provided as the game application 1, an instruction relating to the respiration frequency of the user during the ongoing game is given to the user as the height of the ceiling T in the virtual game world displayed on the monitor 2. The height of the ceiling T is controlled depending on a biological signal (and a biological parameter derived from the biological signal) which has been acquired in advance of the start of the game application 1.

Specifically, for the initial setting in step 71, the game apparatus body 5 reads, with reference to the biological parameter history data Ds22 and the phase data Ds23, an average value of respiration frequencies (hereinafter referred to as “reference respiration frequency”) in a period between the start of acquisition of a biological signal by the vital sensor 76 in step 34 (in FIG. 29) and the start of the game application 1. At this time, the game apparatus body 5 may refer to the biological signal history data Ds21 instead of the biological parameter history data Ds22 to calculate a reference respiration frequency, based on the referred biological signal, with referring to the phase data Ds 23.

Next, the CPU 10 calculates, based on the reference respiration frequency, a respiration frequency at the start of the stretching game, a respiration frequency at the end of the stretching game, and a rate of change from the value at the start of the stretching game to the value at the end of the stretching game, and stores the calculated values in the range setting data Dg.

For example, the CPU 10 configures a respiration frequency change pattern in which respiration starts at a respiration frequency higher than the reference respiration frequency (i.e., faster respiration; e.g., respiration at a speed of 120% relative to the reference respiration), and finally, reaches a respiration frequency lower than the reference respiration frequency (i.e., slower respiration; e.g., respiration at a speed of 80% relative to the reference respiration), and the respiration frequency changes at a predetermined change rate (i.e., the respiration frequency gradually decreases at the predetermined change rate). Then, the CPU 10 stores a parameter that defines the change pattern, in the range setting data Dg. The CPU 10 can control the processes in the following steps, according to the parameter, with reference to the range setting data Dg.

Further, the CPU 10 calculates, with reference to the pulse wave signal of the pulse wave data Dc, a current pulse wave amplitude PA (refer to FIG. 9) which is obtained from the pulse wave signal, as an initial pulse wave amplitude PAi. Then, the CPU 10 updates the initial pulse wave amplitude data Dk using the calculated initial pulse wave amplitude PAi. In some cases, the value calculated as the PAi may be replaced with the average value of the pulse wave amplitudes PA in the “IN” period before the start of the stretching game.

Further, in the initial setting for the stretching game process in step 71, other parameters to be used for the subsequent stretching game process are also initialized. For example, the CPU 10 initializes the score data D1 to a score indicating a perfect score (e.g., 100 points).

Next, in step 72, the CPU 10 obtains, from the core unit 70, data indicating operation information, and goes to the next step.

Specifically, for example, the CPU 10 obtains the received operation information from the core unit 70, and updates the acceleration data Da using the acceleration indicated by the latest acceleration data included in the operation information. More specifically, the CPU 10 updates the X-axis direction acceleration data Da1 using the acceleration indicated by the X-axis direction acceleration data included in the latest operation information received from the core unit 70. Further, the CPU 10 updates the Y-axis direction acceleration data Da2 using the acceleration indicated by the Y-axis direction acceleration data included in the latest operation information. Then, the CPU 10 updates the Z-axis direction acceleration data Da3 using the acceleration indicated by the Z-axis direction acceleration data included in the latest operation information. Further, the CPU 10 updates the key data Db using the operation contents for the operation section 72, which is indicated by the latest key data included in the operation information received from the core unit 70. Furthermore, the CPU 10 updates the pulse wave data Dc using the pulse wave signal indicated by the latest biological information data included in the operation information received from the core unit 70.

Next, in step 73, the CPU 10 calculates an inclination of the core unit 70 with respect to the direction of gravity, and goes to the next step.

For example, the CPU 10 uses an X-axis direction acceleration stored in the X-axis direction acceleration data Da1, a Y-axis direction acceleration stored in the Y-axis direction acceleration data Da2, and a Z-axis direction acceleration stored in the Z-axis direction acceleration data Da3, to calculate an acceleration vector having the acceleration components in the respective directions, and updates the acceleration vector data Dd using the acceleration vector. The CPU 10 also assumes that a direction indicated by the acceleration vector in the acceleration vector data Dd is a direction of a gravitational acceleration acting on the core unit 70. Then, the CPU 10 calculates an inclination of the core unit 70 (an inclination of the controller) with respect to the direction indicated by the acceleration vector, and updates the controller inclination data Dh using the calculated inclination of the core unit 70. Specifically, when the user in the operation posture as shown in FIG. 18 operates the core unit 70, i.e., when it is assumed that the core unit 70 is operated in a manner which inclines the Z axis of the core unit 70 around the X axis direction of the core unit 70, an inclination of the Z axis of the core unit 70 with respect to the direction of the gravitational acceleration is calculated as the inclination of the core unit 70 (the inclination of the controller).

Next, in step 74, the CPU 10 inclines the player character PC with respect to the virtual game world, depending on the inclination of the core unit 70, displays the inclined player character PC on the monitor 2, and goes to the next step.

For example, when it is assumed that the core unit 70 is operated in a manner which inclines the Z-axis of the core unit 70 around the X-axis direction of the core unit 70, then if the user, facing the monitor 2, inclines the core unit 70 so that its Z-axis is inclined to the right at an angle of α, the CPU 10 calculates an inclination angle at which the player character PC inclines to the right at an angle of α in the virtual game world in synchronization with the act of inclining, and updates the inclination data Dq1 using the calculated inclination angle. Thereafter, the CPU 10 inclines the player character PC in the virtual game world, depending on the inclination angle indicated by the inclination data Dq1, and displays the inclined player character PC on the monitor 2 (refer to FIG. 21 and FIG. 22).

Next, in step 75, the CPU 10 calculates a heart rate HR of the user, updates the history of the heart rate data Di using the calculated heart rate HR, and goes to the next step.

For example, the CPU 10 refers to the pulse wave signal of the pulse wave data Dc, and calculates a current cardiac cycle (R-R interval; refer to FIG. 9). Thereafter, the CPU 10 calculates a heart rate HR by dividing 60 sec by the cardiac cycle, and updates the history of heart rates HR by adding data indicating the newly calculated heart rate HR to the heart rate data Di. Note that, as will be seen from a description below, if the history of heart rates HR is stored in an amount corresponding to a predetermined period of time, a process can be performed, and therefore, when a new heart rate HR is added, a past heart rate HR exceeding the time period may be erased.

Next, in step 75, the CPU 10 calculates a pulse wave amplitude PA of the user, updates the history of the pulse wave amplitude data Dj using the calculated pulse wave amplitude PA (step 88), and goes to the next step.

For example, the CPU 10 refers to the pulse wave signal of the pulse wave data Dc to calculate a current pulse wave amplitude PA (refer to FIG. 9) obtained from the pulse wave signal. Thereafter, the CPU 10 adds data indicating the newly calculated pulse wave amplitude PA to the pulse wave amplitude data Dj to update the history of pulse wave amplitudes PA. Note that, as will be seen from a description below, if the history of pulse wave amplitudes PA is stored in an amount corresponding to a predetermined period of time, a process can be performed, and therefore, when a new pulse wave amplitude PA is added, a past pulse wave amplitude PA exceeding the time period may be erased.

Next, in step 77, the CPU 10 determines whether or not the heart rate HR calculated in step 75 is smaller than the previously calculated heart rate HRb.

When the heart rate HR calculated in step 75 is smaller than the previously calculated heart rate HRb (YES in step 77), the CPU 10 goes to step 78.

On the other hand, when the heart rate HR calculated in step 75 is larger than or equal to the previously calculated heart rate HRb (NO in step 77), the CPU 10 goes to step 79.

In step 78, the CPU 10 moves up the second player character PC2 with respect to the first player character PC1 by a predetermined amount in the virtual game world, displays the second player character PC2 thus moved up on the monitor 2, and goes to the next step 81 (refer to FIG. 32B).

For example, the CPU 10 calculates a flying height of the second player character PC2 by increasing the distance between the first player character PC1 and the second player character PC2 in the virtual game world by a predetermined length, and updates the flying height data Dq2 using the flying height. Thereafter, the CPU 10 displays, on the monitor 2, the second player character PC2 being moved up with respect to the first player character PC1 in the virtual game world so that they are separated by the flying height indicated by the flying height data Dq2 (refer to FIG. 20). Note that the distance between the first player character PC1 and the second player character PC2 which are separated in step 78 may be increased by a constant amount, or by an amount which varies depending on a difference between the heart rate HRb and the heart rate HR.

On the other hand, in step 79, the CPU 10 determines whether the heart rate HR calculated in step 75 is larger than or equal to the previously calculated heart rate HRb. When the heart rate HR calculated in step 75 is larger than the previously calculated heart rate HRb (YES in step 79), the CPU 10 goes to step 80. In step 80, the CPU 10 moves down the second player character PC2 with respect to the first player character PC1 by a predetermined amount in the virtual game world, displays the second player character PC2 thus moved down on the monitor 2, and goes to the next step 81 (refer to FIG. 32B).

For example, the CPU 10 calculates a flying height of the second player character PC2 by reducing the distance between the first player character PC1 and the second player character PC2 in the virtual game world by a predetermined length, and updates the flying height data Dq2 using the flying height. Thereafter, the CPU 10 displays, on the monitor 2, the second player character PC2 which is moved up with respect to the first player character PC1 in the virtual game world so that they are separated by the flying height indicated by the flying height data Dq2.

Note that an amount by which the second player character PC2 is moved down with respect to the first player character PC1 in the virtual game world, is decided to a value which prevents the second player character PC2 from overlapping the first player character PC1. In other words, the second player character PC2 is not moved down to a position which causes the second player character PC2 to overlap the first player character PC1 in the virtual game world. Note that the distance between the first player character PC1 and the second player character PC2, which is reduced in step 91, may be decreased by a predetermined amount, or by an amount which varies depending on a difference between the heart rate HRb and the heart rate HR.

When the heart rate HR calculated in step 75 is equal to the previously calculated heart rate HRb (NO in step 79), the CPU 10 goes to the next step 81.

Next, the subsequent processing will be described with reference to FIG. 32B. In step 81, the CPU 10 determines whether or not the player character PC contacts the ceiling T or the ground B in the virtual game world. For example, when the player character is flying, if the first player character PC1 contacts the ground B or the second player character PC2 contacts the ceiling T, the CPU 10 determines that the player character PC contacts the ceiling B or the ground B. Thereafter, the CPU 10, when the player character PC contacts the ceiling T or the ground B, goes to the next step 82 (YES in step 81). On the other hand, if the player character PC contacts none of the ceiling T and the ground B (NO in step 81), the CPU 10 goes to the next step 83.

In step 82, the CPU 10 reduces the score of the stretching game by a predetermined number of points, and goes to the next step 83. For example, the CPU 10 subtracts a point or points corresponding to the contact with the ceiling T or the ground B from the score indicated by the score data Dl, and updates the score data Dl using the resultant score. Here, the number of points to be subtracted may be changed, depending on the situation of contact of the player character PC with the ceiling T or the ground B. As a first example, the number of points to be subtracted is increased according to a period of time during which the player character PC contacts the ceiling T or the ground B. As a second example, the number of points to be subtracted is increased according to an area on which the player character PC overlaps the ceiling T or the ground B. As a third example, the number of points to be subtracted is increased according to the number of times the player character PC contacts the ceiling T or the ground B. As a fourth example, the number of subtracted points is changed, depending on which of the ceiling T and the ground B the player character PC contacts. As a fifth example, the number of subtracted points is changed in accordance with a combination of at least two of the first to fourth examples.

Note that, in the aforementioned process, when the player character PC contacts or overlaps the ceiling T or the ground B, the score of the stretching game is reduced to degrade the assessment. Therefore, the lower the score of the stretching game is, the poorer the assessment is. Alternatively, the score may be changed in other fashions.

As a first example, the score of the stretching game at the start is set to 0 points, and when the player character PC contacts or overlaps the ceiling T or the ground B, the score of the stretching game is incremented to degrade the assessment. In this case, the higher the score of the stretching game is, the poorer the assessment is. As a second example, the score of the stretching game at the start is set to 0 points, and is incremented with time in the stretching game. When the player character PC contacts or overlaps the ceiling T or the ground H in the stretching game, the increment is not performed to degrade the assessment. In this case, the lower the score of the stretching game is, the poorer the assessment is.

In step 83, the CPU 10 calculates a respiration frequency to be indicated to the user, based on the reference respiration frequency obtained in step 71, and goes to the next step. For example, the CPU 10 calculates a value of a respiration frequency to be indicated to the user, with reference to the range setting data Dg. That is, the CPU 10 calculates a respiration frequency to be indicated to the user, according to the respiration frequency variation pattern that is set in step 71. In parallel with the calculation process, the CPU 10 can, as necessary, calculate a current respiration frequency of the user, based on the history of the heart rates HR, and update the respiration frequency data Dm using the calculated respiration frequency. If the heart rate HR calculated in this embodiment is increasing, it is determined that the user is inhaling, and if the heart rate HR is decreasing, it is determined that the user is exhaling.

Next, the CPU 10 calculates a cycle at which the ceiling T is caused to rise and fall (rising/falling frequency), using a parameter that defines the respiration frequency to be indicated to the user, which is calculated in step 83, updates the rising/falling frequency data Dn, and goes to the next step.

Next, in step 85, the CPU 10 calculates an average value PAa of pulse wave amplitudes PA over a predetermined number of pulses, and goes to the following step 86.

Thereafter, in step 86, the CPU 10 determines whether or not the calculated average value PAa is 90% or less of the initial pulse wave amplitude PAi. When PAa≦0.9PAi (YES in step 86), the CPU 10 goes to the next step 87. On the other hand, when 0.9PAi<PAa (NO in step 86), the CPU 10 goes to the next step 92.

In step 87, the CPU 10 sets a color and a countenance of the player character PC displayed in the virtual game world, which represents a level of difficulty, as the situation data Dq3, displays, on the monitor 2, the player character PC in a state corresponding to the difficulty level, and goes to the next step. here, step 87 is executed in a state where the pulse wave amplitude PA of the user is reduced to 90% or less of that at the start of the stretching game, i.e., it can be determined that “the user has difficulty.” When it can be determined that the user has difficulty in the stretching game, the CPU 10 changes the color and the countenance of the player character PC, depending on the level of difficulty/easiness of the user (see FIG. 22).

In step 88, the CPU 10 determines whether or not the calculated average value PAa is 50% or less of the initial pulse wave amplitude PAi. When PAa>0.5PAi (NO in step BB), the CPU 10 goes to the next step 92. When PAa≦0.5PAi (YES in step 88), the CPU 10 goes to the next step 89.

In step 89, the CPU 10 records the current inclination angle of the ground B as a limit inclination angle, and goes to the next step 90. Here, step 89 is executed in a state where the pulse wave amplitude PA of the user is reduced to 50% or less of that at the start of the stretching game, i.e., it can be determined that “the user has much difficulty.” When, it can be determined that the user has much difficulty in the stretching game, the CPU 10 determines that the current inclination angle of the ground B is a limit for the user, and updates the limit ground angle data Dp using the inclination angle.

On the other hand, in step 92, the CPU 10 increases the inclination angle of the ground B by a predetermined angle, and goes to the next step 90. For example, the CPU 10 adds a predetermined angle to an inclination angle indicated by the ground angle data Do to calculate a new inclination angle, and updates the ground angle data Do using the calculated inclination angle.

In step 90, the CPU 10 generates the ceiling T and the ground B (obstacle images) based on the rising/falling frequency indicated by the rising/falling frequency data Dn and the inclination angle indicated by the ground angle data Do, displays the ceiling T and the ground B on the monitor 2, and goes to the next step. For example, the CPU 10 displays, on the monitor 2, the ceiling T which is scrolled while adjusting its shape so that the ceiling T rises and falls at the rising/falling frequency indicated by the rising/falling frequency data Dn, when the player character PC flies in the virtual game world. The CPU 10 also displays, on the monitor 2, the ground B which is inclined at an inclination angle indicated by the ground angle data Do in the virtual game world.

Next, in step 91, the CPU 10 determines whether or not to end the stretching game. For example, the stretching game should be ended when the conditions for game over are satisfied, or when the user performs an operation to end the stretching game. The CPU 10, when not ending the stretching game, returns to step 72 (refer to FIG. 32A) and repeats the process. The CPU 10, when ending the stretching game, ends the subroutine process.

In the above description, the stretching game is described as an example of the game application process defined by step 36 in FIG. 29. As already described, after the process of step 36 is executed, display of the result presentation of a biological parameter such as CVRR acquired before and after the game application process) is performed. Also during this display (presentation) process, biological signals such as pulse wave signals can be acquired from the user via the vital sensor 76 to use the biological signals for the subsequent information processing.

The history of the biological signals and the history of the biological parameters derived from the biological signals may be stored in the account data Ds at a predetermined frequency (e.g., at every one hour from the user's logging into the account in the user determination process (step 34)). Alternatively, those histories having been continuously sampled at every minimum time unit after the login operation may be stored as they are.

In the above description, predetermined presentation is performed using parameters such as a coefficient of variance of R-R interval, a relax fluid amount, a cardiac cycle (R-R interval), a heart rate HR, a respiration frequency, a pulse wave amplitude PA, a difficulty/easiness level, and the like, which are obtained from the biological signal (pulse wave signal) of the user. Alternatively, other parameters acquired from the biological signal (pulse wave signal) of the user may be used.

As a first example, predetermined presentation may be performed using a blood flow rate obtained from the biological signal (pulse wave signal) of the user. For example, the blood flow rate can be obtained by dividing a pulse wave area PWA (refer to FIG. 9) obtained from the pulse wave signal by a heart rate HR.

As a second example, predetermined presentation may be performed using user's tension or activity level (an activity level of the sympathetic nervous system) which are obtained from the biological signal (pulse wave signal). For example, a heart rate HR of the user at rest is compared with a current heart rate HR to calculate user's tension or activity level (e.g., {(current heart rate HR)/(heart rate HR at rest)}×100).

In the above description, a portion (e.g., a fingertip) of the user's body is irradiated with infrared light, and a biological signal (a pulse wave signal) of the user is obtained based on the amount of infrared light which is transmitted through the portion of the body and is sensed, in other words, a change in volume of blood vessels is detected by a so-called optical method to obtain a volume pulse wave. Alternatively, in the present invention, a biological signal of the user may be obtained using sensors of other types which can obtain physiological information that occurs when the user performs a physical activity.

For example, a biological signal of the user may be obtained by detecting (using a piezoelectric method or the like) a change in pressure in blood vessels due to pulsation of arteries to obtain a pressure pulse wave. Alternatively, a muscle potential or heart potential of the user may be obtained as biological information of the user. The muscle or heart potential can be detected by a common detection method using electrodes. For example, a biological signal of the user can be obtained based on, for example, a minute change in current in the user's body. Alternatively, a blood flow of the user may be obtained as biological information of the user. A blood flow is measured as a pulsating blood flow per heartbeat by using an electromagnetic method, an ultrasonic method or the like, and thus measured pulsating blood flow can be used as a biological signal of the user. Of course, the vital sensor 76 may be attached to a portion of the user (e.g., a chest, an arm, an ear lobe, etc.) other than a fingertip in order to obtain the various biological signals described above. Strictly speaking, there may be a difference between pulsation and heartbeat, depending on the obtained biological signal. However, a heart rate and a pulse rate are considered to be substantially equal to each other, and therefore, the obtained biological signal can be processed in a manner similar to the aforementioned process.

In the above description, the vital sensor 76 transmits data indicating a pulse wave signal to the game apparatus body 5, and the game apparatus body 5 calculates various parameters from the pulse wave signal. Alternatively, data in other process steps may be transmitted to the game apparatus body 5. For example, the vital sensor 76 may calculate parameters, such as a CVRR, a relax fluid amount (an activity level of the parasympathetic nervous system), a cardiac cycle (R-R interval), a heart rate HR, a respiration frequency, a pulse wave amplitude PA, an activity level of the sympathetic nervous system, a difficulty/easiness level and the like, and transmit data indicating the parameters to the game apparatus body 5. Alternatively, data in the middle of the calculation of the parameters from the pulse wave signal may be transmitted from the vital sensor 76 to the game apparatus body 5.

In the above description, a current motion and/or posture of the user (a motion of the core unit 70) is detected using an acceleration indicated by triaxial acceleration data obtained from the acceleration sensor 701. Alternatively, a current motion and/or posture of the user may be detected using data outputted from a sensor of another type which is fixed to the core unit 70. For example, it is possible to use data outputted from a sensor (an acceleration sensor or an inclination sensor) which outputs data corresponding to an inclination of the core unit 70 with respect to the direction of gravity (hereinafter simply referred to as “inclination”), a sensor (a magnetic sensor) which outputs data corresponding to an orientation of the core unit 70, a sensor (a gyro-sensor) which outputs data corresponding to a rotational movement of the core unit 70, or the like. The acceleration sensor and the gyro-sensor may be either one which can detect accelerations along multiple axes or one which can detect an acceleration along only a single axis. Alternatively, these sensors may be combined to perform more accurate detection. Note that a camera (e.g., the imaging information calculation section 74) fixed to the core unit 70 can be used as the sensor. In this case, since an image captured by the camera varies depending on a motion of the core unit 70, the motion of the core unit 70 can be determined by analyzing the image.

The sensor may be provided outside the core unit 70, depending on the type thereof. As an example, a camera as the sensor is used to shoot the whole core unit 70 from the outside of the core unit 70, and an image of the core unit 70 included in the captured image is analyzed, whereby the motion of the core unit 70 can be determined. Alternatively, a system including a unit fixed to the core unit 70 and another unit provided outside the core unit 70, which cooperate with each other, may be used. As an example of this system, a light-emitting unit is provided outside the core unit 70, and a camera fixed to the core unit 70 is used to capture light emitted from the light-emitting unit. By analyzing an image captured by this camera, a motion of the core unit 70 can be determined. As another example, a system including a magnetic field generator provided outside the core unit 70 and a magnetic sensor fixed to the core unit 70, may be used.

When the sensor can be provided outside the core unit 70, the core unit 70 may be dispensed with. As an example, a camera as a sensor is used to simply shoot the user, and an image of the user included in the captured image is analyzed, whereby a motion and/or posture of the user can be determined. Alternatively, a sensor which is provided in an input device (e.g., a board controller) that is operated by the user standing thereon, and senses a weight applied to the input device or presence or absence of an object on the input device, can be used to determine a motion and/or posture of the user operating the input device. When these sensors are used to determine a motion and/or posture of the user, the core unit 70 may be dispensed with.

In the above description, a motion of the player character PC, a displayed state of the player character PC, or an image of an obstacle is changed depending on a current biological signal of the user or a current motion and/or posture of the user, to present an image indicating a current condition of the user, an image for prompting the user to change his/her state, or the like. Alternatively, the presentation to the user may be performed in other fashions.

For example, information indicating a current motion and/or posture of the user, an instruction which prompts the user to change his/her state, or the like may be presented using voice, light or the like, depending on a current biological signal of the user and a current motion and/or posture of the user. For example, in the game system 1, voice can be outputted through the loudspeakers 2a or the loudspeaker 706. Specifically, a respiration frequency at which the user should breathe may be indicated to the user by alternately repeating voice sounds “inhale” and “exhale”, instead of presenting the ceiling T which rises and falls. Also, an instruction for the user to act may be performed by repeatedly outputting a voice sound “incline a little more” until a limit inclination angle is reached, and outputting a voice sound “stop now” when the limit inclination angle is reached, instead of presenting the ground B which is inclined.

In the above description, a game is adopted in which the player character PC is moved in the two-dimensional virtual game world, depending on a pulse wave signal of the user and an inclination of the core unit 70. The present invention is applicable to a game in which the player character PC is moved in a three-dimensional virtual game space, and the way of displaying the player character PC is changed.

(Another Example of Game Application Using Biological Information: Shooting Game)

The following will describe more specific processing of a shooting game which can be utilized as a game application in the above-described biological information processing program.

FIG. 33 is a flowchart showing an example of a process of such shooting game. The process shown in this flowchart is, for example, a process to be executed in step 45 among the processes of the biological information processing program shown in FIG. 29.

In step 101, the CPU 10 initializes settings for the game process, and goes to the next step. For example, in the initial setting for the game process in step 101, the CPU 10 performs setting of a virtual game world, and initial settings for a player character PC3, enemy characters EC, and the like. Further, the CPU 10 initializes the respective parameters to be used in the following game process.

In the initial setting performed in step 101, the characteristics on the setting of the game application, which are assigned to the player character operated by the user, can be set using the biological parameters stored in the account data Ds of the user of the game application, regardless of the values possessed by the application of the shooting game as default values.

The characteristics of the player character (the number of bullets to be discharged, the attack power, the discharge direction) on the setting of the game application, which are defined by discharge bullet setting table data Dt (refer to Table 1 as follows) of the shooting game, depend on the heart rate HR of the user based on the pulse wave data obtained from the vital sensor 76. Threshold values X1, X2, and X3 which define the relationship between this heart rate HR, and the number of bullets to be discharged, the attack power, and the discharge direction, can be made dependent on the biological parameters stored in the account data Ds of the user.

For example, in advance of execution of the shooting game in step 45 shown in FIG. 29, the threshold values X1, X2 and X3 are determined based on an average of heart rates (e.g., an average in the “IN” period) obtained in the vital sensor process in step 34, or the game application process defined in step 36, or the result display defined in step 37. For example, when the average is 60, the average in the “IN” period, i.e., 60, is assigned to X2, thereby previously setting the relationships of X1=X2−5 and X3=X2+5. These relational expressions are merely examples, and can be arbitrarily changed depending on the type of the game application to be used.

TABLE 1

| | Number of Bullets | Attack | Discharge |
|---|---|---|---|
| Heart Rate HR | to be Discharged | Power | Direction |
| HR < X1 | 1 | 120 | A |
| X1 ≦ HR < X2 | 2 | 60 | B |
| X2 ≦ HR < X3 | 3 | 40 | C |
| X3 ≦ HR | 5 | 24 | D |

Next, in step 102, the CPU 10 obtains data indicating the operation information from the core unit 70, and goes to the next step. For example, the CPU 10 obtains the operation information received by the core unit 70, and updates the key data Db using the operation contents to the operation section 72, which is indicated by the latest key data included in the operation information. Further, the CPU 10 updates the pulse wave data Do using the pulse wave signal indicated by the latest biological information included in the operation information received by the core unit 70.

Next, in step 103, the CPU 10 moves the player character PC3 in the virtual game world, according to the contents of operation on the operation section 72, which is indicated by the key data Db, and goes to the next step. For example, when the key data Db indicates that the left-side portion of the cross key 72a is pressed, the CPU 10 moves the player character PC3 leftward in the virtual game world by a predetermined distance.

Specifically, the CPU 10 moves the position of the player character PC3, which is indicated by the player character position data Du, leftward in the virtual game world by a predetermined distance, and updates the player character position data Du using the position of the moved player character PC3. On the other hand, when the key data Db indicates that the right-side portion of the cross key 72a is pressed, the CPU 10 moves the player character PC3 right in the virtual game world by a predetermined distance. Specifically, the CPU 10 moves the position of the player character PC3, which is indicated by the player character position data Du, rightward in the virtual game world by a predetermined distance, and updates the player character position data Du using the position of the moved player character PC3.

Next, in step 104, the CPU 10 determines whether or not the current time point corresponds to a pulse timing. When the current time point corresponds to a pulse timing, the CPU 10 goes to the next step. On the other hand, when the current time point does not correspond to a pulse timing, the CPU 10 goes to the next step 108.

For example, in step 104, the CPU 10 detects a specific shape feature point in the pulse wave with reference to the pulse wave signal indicated by the pulse wave data Dc, and when the current time point corresponds to the shape feature point, the CPU 10 determines that the current time point is a pulse timing. As an example of the shape feature point, one of the following points may be selected: a point at which the pulse wave has a minimum value; a point at which the pulse wave has a maximum value; a point at which the speed of vessel contraction is maximum; a point at which the speed of vessel expansion is maximum; a point at which the acceleration of the vessel expansion speed is maximum; and a point at which the deceleration of the vessel expansion speed is maximum. Any of these points may be adopted as the shape feature point to be determined as a pulse timing.

In step 105, the CPU 10 calculates a heart rate HR of the user, updates the heart rate data Di using the calculated heart rate HR, and goes to the next step.

For example, the CPU 10 calculates, with reference to the pulse wave signal indicated by the pulse wave data Dc, a time interval (e.g., R-R interval; refer to FIG. 9) from the previously detected pulse timing to the pulse timing currently detected in step 104, as a cardiac cycle at the current time point. Then, the CPU 10 divides 60 seconds by the cardiac cycle to calculate a heart rate HR, and updates the heart rate data Di using the newly calculated heart rate HR. Note that, when a pulse timing is detected for the first time in this process, the CPU 10 updates the heart rate data Di using a predetermined constant (e.g., 0) as a heart rate HR.

Next, in step 106, the CPU 10 sets the number of bullets to be discharged and the attack power, based on the heart rate HR calculated in step 105.

For example, the CPU 10 extracts, with reference to the discharge bullet setting table data Dt, the “number of bullets to be discharged” and the “attack power” corresponding to the heart rate HR calculated in step 105. Then, the CPU 10 sets the number corresponding to the extracted “number of bullets to be discharged” as the number of bullets B to be discharged per pulse timing, and sets the extracted “attack power” as the attack power corresponding to each bullet B to be discharged.

Next, in step 107, the CPU 10 performs a process of causing the player character PC3 to discharge the bullets B set in step 106 toward the set discharge direction, and goes to the next step. Specifically, the CPU 10 causes the bullets B, the number and attack power of which have been set in step 106, to newly appear in the virtual game world, and causes the player character PC3 to discharge the bullets B toward the “discharge directions” corresponding to the respective bullets B.

In step 108, the CPU 10 performs a control for operating other objects in the virtual game world, based on predetermined operation rules, and goes to the next step.

For example, the CPU 10 causes the enemy characters EC which have already been arranged in the virtual game world, to move in predetermined directions by predetermined distances, causes new enemy characters EC to appear in the virtual game world, and causes the enemy characters EC to disappear from the virtual game world when the enemy characters EC have been hit by the bullets B. Then, the CPU 10 updates the enemy character position data By according to the respective situations. Further, the CPU 10 causes the bullets B which have already been discharged in the virtual game world to move along the set “discharge directions” by predetermined distances, and causes the bullets B to disappear from the virtual game world when the bullets B have hit the enemy characters. Then, the CPU 10 updates the bullet object position data Dw.

Next, in step 109, the CPU 10 performs a process of displaying, on the monitor 2, the virtual game world in which the player character PC3, the enemy characters EC, the bullets B, and the like are arranged, and goes to the next step. For example, the CPU 10 arranges the player character PC3, the enemy characters EC, the bullets B, and the like in the virtual game world, by using the player character position data Du, the enemy character position data Dv, the bullet object position data Dw, and the image data Dx, and then performs a process of displaying a predetermined range in the virtual game world on the monitor 2.

Next, in step 110, the CPU 10 determines whether or not the game should be ended. The game is ended when, for example, the conditions for game over are satisfied, or the user performs an operation to end the game. When the game should not be ended, the CPU 10 returns to step 102 to repeat the process. When the game should be ended, the CPU 10 ends the processing of the flowchart.

(Modifications)

In the above-described examples, the present invention is applied to the stationary game apparatus 3. The present invention is also applicable to any apparatus that includes at least a vital sensor 76, a sensor, such as an acceleration sensor, an inclination sensor, or the like, for detecting a motion and/or posture of the user, and an information processing device for executing processes depending on information obtained from these sensors. For example, the present invention is also applicable to general devices, such as a personal computer, a cellular phone, a personal digital assistant (PDA), a hand-held game apparatus, and the like.

As another embodiment, the information processing program of the present invention may be executed in such a mode (distributed system) that individual parts constituting the information processing program are executed on a plurality of computers simultaneously and in parallel with each other, and the individual parts are allowed to mutually communicate via a network. For example, when a virtual game world is set on another apparatus, data in the middle stage of the above-described game process may be transferred from the game apparatus 3 to the other apparatus, and after the other apparatus has performed a process using the transferred data, the game apparatus 3 may perform a display process. In this way, by performing, in another apparatus, at least part of the process steps of the game process, a process similar to the above-described game process can be realized. Moreover, the present invention can be applied to a game process in which players of a plurality of game apparatuses participate in a virtual game world realized on another apparatus.

In the above description, the core unit 70 and the game apparatus body 5 are connected by wireless communication. Alternatively, the core unit 70 and the game apparatus body 5 may be electrically connected via a cable. In this case, a cable connected to the core unit 70 is connected to a connection terminal of the game apparatus body 5.

Further, of the core unit 70 and the vital sensor 76 constituting the controller 7, the communication section 75 is provided only in the core unit 70. Alternatively, a communication section which wirelessly transmits biological information data to the game apparatus body 5 may be provided in the vital sensor 76. Still alternatively, the communication section may be provided in each of the core unit 70 and the vital sensor 76. For example, the communication sections provided in the core unit 70 and the vital sensor 76 may each wirelessly transmit biological information data or operation data to the game apparatus body 5. Alternatively, the communication section of the vital sensor 76 may wirelessly transmit biological information data to the core unit 70, and the communication section 75 of the core unit 70 may receive it and then wirelessly transmit operation data of the core unit 70 along with the biological information data of the vital sensor 76 to the game apparatus body 5. In these cases, the connection cable 79 for electrically connecting the core unit 70 and the vital sensor 76 is no longer required.

Further, the shape of the core unit 70 and the shapes, number, arrangement and the like of the operation sections 72 provided thereon, which are described above, are only for illustrative purposes. Even in the case of using other shapes, numbers, arrangements and the like, the present invention can be achieved. Also, the shape of the vital sensor 76 and the types, number, arrangement and the like of the components provided therein, which are described above, are only for illustrative purposes. Even in the case of using other types, numbers, arrangements and the like, the present invention can be achieved. Also, the aforementioned coefficients, criteria, expressions, procedures and the like used in the above-described processes are only for illustrative purposes. Even in the case of other values, expressions and procedures, the present invention can be achieved.

Further, the information processing program of the present invention may be supplied to the game apparatus body 5 not only from an external storage medium such as the optical disc 4 or the like, but also via a wireless or wired communication line. Alternatively, the information processing program may be previously stored in a nonvolatile storage device of the game apparatus body 5. Examples of the information storage medium that stores the information processing program include a CD-ROM, a DVD, an optical disc-like storage medium similar to those, and a nonvolatile semiconductor memory.

Note that, in this specification, in order to simply exemplify the features of the present invention, the program for acquiring a biological signal is separated from other game applications. However, this configuration is merely an example. Accordingly, the functions provided to the user by the whole biological information processing program of the present invention can be reconfigured using constituent units of another program, while maintaining the essence of the features of the biological information processing program exemplified in this specification (for example, modularization of predetermined functions can be utilized).

Recently, as a general trend, operating systems of typical computers are composed from various separate parts, called modules or functions; and offer a number of services via the separate parts. Further, in a commonly employed scheme, an application program calls such modules in a prescribed sequence, if needed, for providing its service. In addition, software for general platforms is not always accompanied with such modules as a package; that is, it can be distributed without modules which are provided commonly in such platforms. Thus, software would be provided, which have been distributed without some modules or functions during the course of distribution thereof; and can offer a service which falls into the scope of the present invention, in connection with modules implemented in a computer executing the software. Such software can be distributed in various ways such as via computer network, in form of a storage medium having such software stored therein, and the like. Therefore, software or program deliberately provided in the above-mentioned manner, should be contemplated to fall into the scope of the substantial portion of the present invention.

A storage medium is provided having stored therein an information processing program relating to biological information, an information processing method, an information processing system, and the like, which are capable of providing information processes adapted for the individual characteristics of users reacting in different ways against a variety of daily environments.

While the invention has been described in detail, the foregoing description is in all aspects illustrative and not restrictive. It is to be understood that numerous other modifications and variations can be devised without departing from the scope of the invention. It is also to be understood that the scope of the invention is indicated by the appended claims rather than by the foregoing description. It is also to be understood that the detailed description herein enables one skilled in the art to make changes coming within the meaning and equivalency range of the present invention. It is also to be understood that all of the patents, patent applications and publications recited herein are hereby incorporated by reference as if set forth in their entirety herein. Furthermore, it should be understood throughout the present specification that expression of a singular form includes the concept of their plurality unless otherwise mentioned. Specifically, articles or adjectives for a singular form (e.g., “a”, “an”, “the”, etc. in English) include the concept of their plurality unless otherwise mentioned. It should be also understood that the terms as used herein have definitions typically used in the art unless otherwise mentioned. Thus, unless otherwise defined, all scientific and technical terms have the same meanings as those generally used by those skilled in the art to which the present invention pertain. If there is contradiction, the present specification (including the definitions) precedes.

What is claimed is:

1. A computer readable storage medium having stored therein an information processing program executable by a computer of an information processing device which performs predetermined processes based on biological parameters acquired from a user, the information processing program causing the computer to function as:
first biological parameter acquisition means for acquiring a first biological parameter from the user during a first period;
second biological parameter acquisition means for acquiring a second biological parameter from the user, within the first period, during the acquisition of the first biological parameter, or on and after a first time point after the acquisition of the first biological parameter; and
first process execution means for executing a first process from the first time point, based on the first biological parameter and the second biological parameter.

2. The computer readable storage medium according to claim 1, wherein
the information processing program causes the computer to further function as:
biological signal acquisition means for acquiring a biological signal from the user;
the first biological parameter acquisition means acquires the first biological parameter based on the biological signal acquired by the biological signal acquisition means; and
the second biological parameter acquisition means acquires the second biological parameter based on the biological signal acquired by the biological signal acquisition means.

3. The computer readable storage medium according to claim 2, wherein
the biological signal acquisition means acquires the biological signal from the user during a second period including the first period.

4. The computer readable storage medium according to claim 2, wherein
the biological signal acquisition means acquires, as the biological signal, a signal relating to pulse or heartbeat of the user.

5. The computer readable storage medium according to claim 2, wherein
the biological signal acquisition means acquires, as the biological signal, at least one selected from the group consisting of a pulse wave, a heart rate, an activity level of sympathetic nervous system, an activity level of parasympathetic nervous system, a coefficient of variance of R-R interval, a cardiac cycle, a respiration frequency, and a pulse wave amplitude of the user.

6. The computer readable storage medium according to claim 1, wherein
the first process execution means executes the first process from the first time point, based on the first biological parameter and the second biological parameter which are acquired during a period corresponding to at least part of a period before the first time point.

7. The computer readable storage medium according to claim 2, wherein
the first process execution means executes the first process, based on the first biological parameter and the second biological parameter, the first biological parameter being based on the biological signal acquired during a period between a time point at which the acquisition of the biological signal is started by the biological signal acquisition means, and the first time point.

8. The computer readable storage medium according to claim 1, wherein
the information processing program causes the computer to further function as:
menu selection means for causing the user to select a desired process from the group consisting of one or more processes including the first process;
the first time point is a point at which the user selects a desired process in the menu selection means; and
the menu selection means causes the user to select a desired process by the first time point.

9. The computer readable storage medium according to claim 8, wherein
the menu selection means causes the user to select a desired process during the first period.

10. The computer readable storage medium according to claim 1, wherein
the first process execution means includes first presentation means for presenting a result of the execution of the first process.

11. The computer readable storage medium according to claim 10, wherein
the first time point is a predetermined point during the acquisition of the first biological parameter; and
the first presentation means presents the result of the execution of the first process, based on the first biological parameter which is acquired when the first presentation means presents the result of the execution of the first process.

12. The computer readable storage medium according to claim 1, wherein
the information processing program causes the computer to further function as:
login means for performing a login process which causes the user to log in; and
logout means for performing a logout process which causes the user to log out, after the login process by the login means; and
the first biological parameter acquisition means acquires the first biological parameter from the user, during a period corresponding to at least part of a period between the login process by the login means and the logout process by the logout means.

13. The computer readable storage medium according to claim 12, wherein
the first biological parameter acquisition means acquires the first biological parameter from the user at predetermined intervals.

14. The computer readable storage medium according to claim 12, wherein
the first biological parameter acquisition means does not acquire the first biological parameter during a period in which the first process execution means executes the first process.

15. The computer readable storage medium according to claim 12, wherein
the first process execution means executes the first process, based on the first biological parameter which has been acquired by the first biological parameter acquisition means before the execution of the first process.

16. The computer readable storage medium according to claim 15, wherein
the first process execution means executes the first process, based on a predetermined number of the first biological parameters which are acquired by the first biological parameter acquisition means.

17. The computer readable storage medium according to claim 1, wherein
the information processing program causes the computer to further function as:
history storage means for storing first biological information in association with the user, the first biological information including the first biological parameter acquired by the first biological parameter acquisition means, or the second biological parameter acquired by the second biological parameter acquisition means, or a combination of them.

18. The computer readable storage medium according to claim 17, wherein
the first process execution means executes the first process also based on the first biological information.

19. The computer readable storage medium according to claim 17, wherein
the information processing program causes the computer to further function as:
second process execution means for executing a second process, based on the first biological information, after the first process.

20. The computer readable storage medium according to claim 1, wherein
the information processing program causes the computer to further function as:
motion/posture information acquisition means for acquiring information about a motion and/or posture of the user, from detection means for detecting this information; and
the first process execution means executes the first process also based on the information acquired by the motion/posture information acquisition means.

21. The computer readable storage medium according to claim 20, wherein
the first process execution means prompts the user to take a predetermined motion and/or posture.

22. The computer readable storage medium according to claim 1, wherein
the first process execution means further performs a process for changing the manner of presentation to the user, depending on the first biological parameter, the second biological parameter, and the periods in which the respective biological parameters are acquired.

23. An information processing device for performing predetermined processes based on a biological parameter acquired from a user, the apparatus comprising:
first biological parameter acquisition means for acquiring a first biological parameter from the user during a first period;
second biological parameter acquisition means for acquiring a second biological parameter from the user, within the first period, during the acquisition of the first biological parameter, or on and after a first time point after the acquisition of the first biological parameter; and
first process execution means for executing a first process from the first time point, based on the first biological parameter and the second biological parameter.

24. An information processing method for performing predetermined processes based on a biological parameter acquired from a user, the method comprising:
a step of executing a first process from a first time point, based on a first biological parameter acquired from the user during a first period, and a second biological parameter which is acquired from the user, within the first period, during the acquisition of the first biological parameter, or on and after a first time point after the acquisition of the first biological parameter.

25. An information processing system including a plurality of devices which are configured to be communicable with each other, and performing predetermined processes based on a biological signal acquired from a user, the system comprising:
first biological parameter acquisition means for acquiring a first biological parameter from the user during a first period;
second biological parameter acquisition means for acquiring a second biological parameter from the user, within the first period, during the acquisition of the first biological parameter, or on and after a first time point after the acquisition of the first biological parameter; and
first process execution means for executing a first process from the first time point, based on the first biological parameter and the second biological parameter.
